(19) United States
(12) Patent Application Publication
(10) Pub. No.: US 2002/0022030 A1
(43) Pub. Date: Feb. 21, 2002

(54) Product and process for regulation of T cell responses

(76) Inventors: Philippa Marrack, Denver, CO (US); John W. Kappler, Denver, CO (US); Chia Chi Ku, Mountain View, CA (US); Masaaki Murakami, Denver, CO (US); Akemi Sakamoto, Inage-ku (JP)

Correspondence Address: Angela Dallas-Pedretti, SHERIDAN ROSS P.C., Suite 1200, 1560 Broadway, Denver, CO, 80202-5141, US

(21) Appl. No.: 09/844,928

(22) Filed: Apr. 26, 2001

Related U.S. Application Data
(60) Provisional application No. 60/199,763, filed on Apr. 26, 2000.

(51) Int. Cl. A61K 39/395; A61K 38/20

(52) U.S. Cl. 424/145.1; 424/85.2

(57) ABSTRACT

Disclosed is a method for increasing a memory T cell response by administering to a patient a composition that increases the activity of IL-15 and that decreases the activity of IL-2. Also disclosed is a vaccine adjuvant useful for increasing memory T cell responses. A method for inhibiting undesirable immune responses, such as autoimmune responses, is also disclosed, as is a composition useful in such a method. The composition includes a compound that increases the activity of IL-2 and in one embodiment, further includes a compound that decreases the activity of IL-15.

### CROSS-REFERENCE TO RELATED APPLICATIONS

This application claims priority under 35 U.S.C. § 119(e) from U.S. Provisional Application Serial No. 60/199,763, filed Apr. 26, 2000, and entitled “Product and Process for Regulation of T Cell Responses”. The entire disclosure of U.S. Provisional Application Serial No. 60/199,763 is incorporated herein by reference.

### FIELD OF THE INVENTION

This invention generally relates to the differential regulation of IL-15 and IL-2 activity to regulate immune responses in an animal, and particularly, to regulate memory T cell responses. In particular embodiments, the invention relates to a composition and method for enhancing vaccination, and to a composition and method for inhibiting autoimmune responses.

### BACKGROUND OF THE INVENTION

Immune responses have memory. Because of this, people do not usually get chicken pox more than once, and vaccination with, for example, polio virus prevents subsequent infection with the same virus. Immunological memory depends on two phenomena. The first exposure to the antigen causes individuals to make antibodies against the antigen. These antibodies persist in the individual for a long time and, when the antigen enters the host subsequently, binding to those antibodies inactivates the antigen in various ways and prevents infection. Second, the first exposure to antigen causes individuals to make memory T cells which can recognize the antigen. These memory T cells respond more quickly and effectively than naive cells, hence they attack the antigen the next time it enters the body more quickly and thus help to prevent second infections with the same invader.

Although persistent antigen may help preserve memory T cell numbers (Gray and Matzinger, J. Exp. Med. 174:969 (1991)), it is now clear that antigen is not needed for memory T cell survival (Lau et al., Nature 369:648 (1994); Mullbacher, J. Exp. Med. 179:317 (1994); Bruno et al., Immunity, 2:37 1995); Bruno et al., Eur. J. Immunol. 26:3179 (1996); Murali-Krishna et al., Science 286:1377 (1999); Swain et al., Science 286:1381 (1999)). Thus memory T cells might not need external stimuli for survival. However, memory T cells and T cells with memory phenotype continue to divide, albeit slowly, in the absence of antigen (Bruno et al., Eur. J Immunol. 26:3179 (1996); Murali-Krishna et al., Science 286:1377 (1999); Swain et al., Science 286:1381 (1999); Tough and Sprent, J. Exp. Med. 179:1127 (1994); Zhang et al., Immunity 8:591 (1998)). This suggests that memory T cells might depend on some constantly available factor(s) to preserve themselves. Prior to the present invention, however, the factors contributing to the preservation of memory T cells were not known.

In several cases of human treatment, the ability to harness memory T cells limits treatment of the disease. For example, current strategies for vaccination against HIV are unsuccessful, in large part because T cell and antibody responses are not large enough to prevent successful infection by all HIV to which an individual is exposed. Conversely, an inability to destroy autoimmune T cells prevents proper treatment of individuals suffering from diseases such as systemic lupus erythematosus and rheumatoid arthritis.

### SUMMARY OF THE INVENTION

One embodiment of the present invention relates to a vaccine adjuvant. The adjuvant includes: (a) an agent that increases interleukin-15 (IL-15) activity; and, (b) an agent that decreases interleukin-2 (IL-2) activity.

The agent that increases IL-15 activity is preferably an agent that increases IL-15 receptor activity without enhancing IL-2 receptor activity. In one aspect, the agent that increases IL-15 activity is IL-15 or a homologue of IL-15 that has IL-15 biological activity. In another aspect, the agent that increases IL-15 activity is an antibody that selectively binds to and activates an IL-15 receptor and does not substantially bind to and activate an IL-2 receptor. In another aspect, the agent selectively binds to IL-15Rα. In yet another aspect, the agent that increases IL-15 activity is an agent that binds to and increases the half-life of IL-15. In another aspect, the agent that increases IL-15 activity is a recombinant nucleic acid molecule comprising a nucleic acid sequence encoding IL-15 or a homologue of IL-15 that has IL-15 biological activity. In another aspect, the agent that increases IL-15 activity is an agent that binds to a regulatory region of a gene encoding IL-15 and increases transcription of the gene encoding IL-15.

In one aspect of this embodiment, the agent that decreases IL-2 activity is an antibody that selectively binds to IL-2 and blocks IL-2, eliminates IL-2 or prevents the interaction of IL-2 with its receptor. In another aspect, the agent that decreases IL-2 activity is a compound that binds to and degrades IL-2. In yet another aspect, the agent that decreases IL-2 activity is a compound that blocks or decreases the activity of IL-2 receptors without blocking or decreasing the activity of IL-15 receptors. In another aspect, the agent selectively binds to IL-2Rα. In yet another aspect, the agent that decreases IL-2 activity is an antisense nucleic acid molecule that hybridizes to a gene encoding IL-2 under high stringency conditions and inhibits the expression of IL-2.

In one embodiment, the vaccine adjuvant further includes a delivery vehicle that is targets memory T lymphocytes. For example, such a delivery vehicle can include an antibody that selectively binds to memory T lymphocytes.

One embodiment of the present invention relates to a vaccine that includes: (a) the vaccine adjuvant as described above; and (b) a vaccinating antigen. Preferably, the vaccinating antigen is selected from the group of: a tumor antigen and an antigen from an infectious disease pathogen. In one embodiment, such a vaccine is used in a method to increase T lymphocyte memory against an antigen by administering the vaccine to an animal.

Another embodiment of the present invention relates to a method to increase T lymphocyte memory. The method includes the step of administering to an animal a composition comprising an agent that increases IL-15 activity and an agent that decreases IL-2 activity. Preferably, the step of administering increases the activity or survival of CD25⁺ T cells in the animal. In one embodiment, the composition is administered to a site of a vaccination in the animal. In another embodiment, the method further includes a step of administering to the animal an antigen against which T lymphocyte memory is to be induced. In this embodiment of the invention, the agent that increases IL-15 activity can be any agent that increases IL-15 activity as previously described above. Similarly, the agent that decreases IL-2 activity can be any agent that decreases IL-2 activity as described above.

Yet another embodiment of the present invention relates to a method to reduce an autoimmune response. This method includes the step of administering to a site of an autoimmune response a composition comprising an agent that increases the activity of IL-2. The agent is preferably an agent that increases IL-2 receptor activity without enhancing IL-15 receptor activity. Such agents can include, but are not limited to, IL-2 or a homologue of IL-2 that has IL-2 biological activity; an antibody that selectively binds to and activates an IL-2 receptor and does not substantially bind to and activate an IL-15 receptor; an agent that selectively binds to IL-2Rα; an agent that binds to and increases the half-life of IL-2; a recombinant nucleic acid molecule comprising a nucleic acid sequence encoding IL-2 or a homologue of IL-2 that has IL-2 biological activity; and/or an agent that binds to a regulatory region of a gene encoding IL-2 and increases transcription of the gene encoding IL-2.

In one aspect, the method further includes the step of administering to the site of the autoimmune response an agent that decreases IL-15 activity. The agent that decreases IL-15 activity can include, but is not limited to: an antibody that selectively binds to IL-15 and blocks IL-15, eliminates IL-15 or prevents the interaction of IL-15 with its receptor; a compound that binds to and degrades IL-15; a compound that blocks or decreases the activity of IL-15 receptors without blocking or decreasing the activity of IL-2 receptors; an agent that selectively binds to IL-15Rα; and/or an antisense nucleic acid molecule that hybridizes to a gene encoding IL-15 under high stringency conditions and inhibits the expression of IL-15.

In one aspect of this method, the composition comprises a delivery vehicle that selectively targets a site of an autoimmune response. For example, such a delivery vehicle can include an antibody that selectively binds to a cell surface molecule expressed by a cell at the site of the autoimmune response. In one aspect, the composition further comprises an autoantigen against which the autoimmune response is directed.

Another embodiment of the present invention relates to a composition for decreasing an undesirable T cell response. The composition includes: (a) an agent that increases the activity of IL-2; and (b) an agent that decreases the activity of IL-15.

### BRIEF DESCRIPTION OF THE DRAWINGS OF THE INVENTION

FIGS. 1A-1D show that memory CD8+ T cells bear high levels of CD44 and IL-2Rβ.

FIGS. 2A-2F shows that T cells with memory phenotype divide slowly in animals.

FIGS. 3A-3B show that the appearance of proliferating CD8+ memory phenotype cells is stimulated by IL-15 and inhibited by IL-2.

FIGS. 4A-B show that the CD8+ T cells stimulated to divide by inhibition of IL-2 are of memory phenotype.

### DETAILED DESCRIPTION OF THE INVENTION

The present invention generally relates to a product and process for the regulation of T cells and T cell-mediated immune responses. Specifically, the present invention generally relates to a product and process for the regulation of T cell memory, and more particularly, to methods of regulating T cell responses under conditions wherein it is desirable to decrease (i.e., inhibit, downregulate, reduce) a T cell response, or alternatively, under conditions wherein it is desirable to increase (i.e., enhance, upregulate, stimulate) a T cell response. A wide variety of medical treatments require regulation of the immune response in a patient. Such treatments include, for example, vaccinations, treatments for autoimmune diseases, immunodeficiency diseases, immunoproliferative diseases, treatments for cancer, and treatments involving the transplantation of organs and skin. The present invention is particularly directed to methods of regulating autoimmune T cell responses (i.e., regulation of autoreactive T cells) and to methods of regulating T cell responses to a specific immunogen (e.g., in a vaccination protocol). Other types of T cell responses which may be regulated by a method of the present invention include, but are not limited to, immunodeficiency diseases (wherein it is desirable to increase T cell activity), immunoproliferative diseases (wherein it is desirable to decrease T cell activity), treatments for cancer (wherein it is desirable to increase T cell activity), and treatments involving the transplantation of organs and skin (wherein it is desirable to decrease T cell activity).

Until the discoveries by the present inventors regarding the relationship between interleukin-2 (IL-2) and interleukin- 15 (IL-15) and T cell responses, it was not known how memory T cells survive and are controlled. The present inventors have shown that memory T lymphocytes (T cells) are kept alive and divide in response to IL-15 and that they are destroyed by IL-2. Thus, the number of memory T cells sustained with in an individual is controlled by the balance of IL-15 and IL-2.

The present inventors' finding teaches that memory responses can be improved, and hence indicates that vaccination and rejection of invading organisms by increasing the activity of IL-15 or its mimics, and/or by decreasing the activity of IL-2 or its mimics will be beneficial. Conversely, in autoimmune diseases, the activity of autoimmune T cells can be reduced by decreasing the activity of IL-15 or its mimics and/or by increasing the activity of IL-2 or its mimics.

No strategy of this type is currently in use. In fact IL-2 is currently being used to increase immune reactivity, exactly the opposite strategy from that taught in the present invention. Moreover, while interleukin-15 has been shown to be immunostimulatory and even suggested for use as an adjuvant (See U.S. Pat. No. 5,747,024 to Grabstein et al.), until the present invention, the effects of IL-15 on the slow division of memory T lymphocytes was not known, nor were the differential effects of IL-15 and IL-2 on immune regulation.

Interleukin 15 is constitutively produced in animals (Tough and Sprent, J. Exp. Med. 179:1127 (1994); Zhang et al., Immunity 8:591 (1998); Peschon et al., J. Exp. Med. 180:1955 (1994); Moore et al., J. Immunol. 157:2366 (1996); Sudo et al., J. Exp. Med. 170:333 (1989); Heufler et al., J. Exp. Med. 178:1109 (1993); Doherty et al., J. Immunol. 1556:735 (1996); Jonuleit et al., J. Immunol. 158:2610 (1997); Tagaya et al., Proc. Natl. Acad. Sci. USA 94:14444 (1997); Bamford et al., J. Immunol. 160:4418 (1998)). Although IL-2 is not constitutively produced in animals, recent evidence suggests that it is present, even in young pathogen free mice, retained on the extracellular matrix (Wrenshall and J. Immunol. 163:3793 (1999)). This may be the source of the IL-2 which is functioning in the experiments reported here. Interleukin-2 can induce activated T cells to die (Zheng et al., J. Immunol. 160:763 (1998); Refaeli et al., Immunity, 8:615 (1998)) and/or, as illustrated by the experiments reported here, kill proliferating CD8+ memory phenotype cells (but see Ke et al., J. Exp. Med. 187:49 (1998)). IL-2 or IL-2Rα deficient mice suffer from lymphoproliferative diseases, especially if infected (Kramer et al., Eur. J. Immunol. 24:2317 (1994); Simpson et al., Eur. J. Immunol. 25:2618 (1995); Willerford et al., Immunity 3:521 (1995); Kung et al., Cell. Immunol. 185:158 (1998); Erhardt et al., J. Immunol. 158:566 (1998)). Without being bound by theory, the present inventors suggest that this is because lack of IL-2 allows unchecked proliferation of memory T cells in response to IL-15 in these animals.

Mice deficient in IL-15Rα lack CD8+ memory phenotype T cells (Lodolce et al., Immunity 9:669 (1998)) and IL-15, induced by poly IC or interferon, makes CD8+ T cells of memory phenotype divide (Tough and Sprent, J. Exp. Med. 179:1127 (1994); Zhang et al., Immunity 8:591 (1998); Tough et al., Science 272:1947 (1996)). However, the experiments described here are the first to suggest that the slow division of memory phenotype CD8+ T cells in specific pathogen free mice is caused by the same cytokine. Competition for IL-15 may, in fact, limit the total number of CD8+ memory CD8+ T cells the animal can sustain (Selin et al., J. Exp. Med. 183:2489 (1996)). Conversely, production of IL-2 during an immune response may check otherwise uncontrolled responses by bystander CD8+ memory T cells induced by increased levels of IL-15.

In immune responses the stimulatory effects of one process are frequently counterbalanced by the inhibitory effects of another. Such contrary effects allow the immune system to respond vigorously but not uncontrollably to infections. The opposing effects of IL-15 and IL-2 reported here represent another example of the checks and balances inherent in the mechanisms of immunity.

Accordingly, one embodiment of the present invention relates to a composition and method for increasing a desirable immune response, and particularly, for enhancing T cell memory in an individual. For example, it is desirable to increase (e.g., enhance, upregulate, stimulate, activate) T cell memory responses in a patient that has cancer (i.e., increase memory T cell responses against a tumor antigen), in a patient with an infectious disease (i.e., increase memory T cell responses against a pathogen, such as a virus or bacterium), and/or in a patient that has an immunodeficiency disease (i.e., increase memory T cell responses against a variety of antigens). Other diseases and conditions in which it is desirable to increase T cell memory will be apparent to those of skill in the art and are intended to be encompassed by the present invention.

Preferably, the memory T cell response is enhanced by administering to the patient a composition comprising at least one agent that increases the activity of IL-15 in the patient and/or at least one agent that decreases the activity of IL-2 in the patient. In a preferred embodiment, both agents are administered together in a composition with or without an antigen against which the memory T cell response is to be increased. When the composition of the present invention is administered in conjunction with an antigen (an immunogen), the composition of the present invention serves as a vaccine adjuvant, to enhance the development of a memory T cell response against the antigen. In a particularly preferred embodiment, the administration of the composition is targeted to a particular site or cell in a patient (e.g., a site of a tumor, an organ that is infected with a pathogen), so that the effect of the composition is substantially localized to the T cells for which increased response is desired.

Specifically, in one aspect, the method of the present invention includes decreasing the action (i.e., the activity) of interleukin-2 (IL-2) in a patient in which enhancement of a T cell immune response is desired. Reference to decreasing the action (or activity) of IL-2 refers to any manipulation of the patient to be treated and specifically, of a cell of a patient to be treated, which results in decreased functionality of IL-2 in the patient, including decreased activity of IL-2 by acting on endogenous IL-2 or the receptor for IL-2 (e.g., by administration of an antibody that specifically binds to and blocks the activity of IL-2 or results in elimination of IL-2; or by administration of an antibody that specifically prevents the interaction of IL-2 with its receptor, preferably without inhibiting the interaction of IL-15 with its receptor; by administering a compound that decreases endogenous IL-2 production; by administering a compound that decreases IL-2 receptor sensitivity or responsiveness in the cells of the patient without decreasing IL-15 receptor responsiveness in a patient; or by increasing degradation of IL-2 in the patient). IL-2 biological activity and methods for evaluating the same are discussed in detail below.

In one aspect of this method of the present invention, the action of IL-2 is decreased in the patient in a manner effective to regulate the activity and/or survival of CD25⁺ T cells in the patient. CD25⁺ T cells are described, for example, in Thornton et al., 2000, J. Immunol. 164(1):183-190, incorporated herein by reference in its entirety. Preferably, the activity of CD25⁺ T cells in the patient is inhibited or diminished, such that the CD25⁺ T cells and more particularly, such that the CD25⁺ T cells have a decreased ability to suppress the responses of CD25⁻ T cells in the patient which, without being bound by theory, can allow for increased proliferation of memory T cells. In a preferred embodiment, the action of IL-2 is decreased in the patient in a manner effective to regulate the activity of CD25⁺ T cells in the patient such that the activity of CD25⁻ T cells, and particularly CD25⁻ T cells for which activity is to be enhanced, is upregulated. More preferably, the activity of CD25⁻ T cells that are recruited to the site of a desirable immune response (e.g., a vaccination site, a cancer site) are upregulated by the method of the present invention. In one embodiment, the effects of IL-2 and of regulation of CD25⁺ T cells are directed to the site of a vaccination or organ or lymph node near the site of a vaccination by administration of the product for the downregulation of IL-2 with the vaccination.

In one aspect of this method of the present invention, the method of upregulating a T cell response includes the step of upregulating the action (i.e., the activity) of interleukin-15 (IL-15) in the patient, in addition to decreasing the action of IL-2. Reference to upregulating or increasing the action (or activity) of IL-15 refers to any manipulation of the patient to be treated and specifically, of a cell of a patient to be treated, which results in increased functionality of IL-15 in the patient, including increasing the activity of IL-15 by acting on endogenous IL-15 or the receptor for IL-15, or by providing exogenous IL-15 or a compound that has IL-15 activity (e.g., by administration of exogenous IL-15 or a mimetic thereof, overexpression of IL-15 in the cells of the patient, administration of a compound that enhances endogenous IL-15 production, administration of a compound that enhances IL-15 receptor sensitivity or responsiveness in the cells of the patient, preferably without enhancing IL-2 receptor sensitivity; or by decreasing degradation of IL-15 in the patient). IL-15 biological activity and methods for evaluating the same are discussed in detail below.

Upregulating a T cell response in an animal can be an effective treatment for a wide variety of medical disorders, and in particular, for cancer and/or infectious disease. As used herein, the term “upregulate” can be used interchangeably with the terms “increase”, “activate”, “stimulate”, “generate” or “elicit”. According to the present invention, “upregulating a T cell response” in an animal refers to specifically controlling or influencing the activity of the T cell response, and can include activating the T cell response, increasing the T cell response, and/or enhancing the T cell response, and more particularly, a memory T cell response.

Preferably, the method of the present invention increases a memory T cell response against a tumor or an infectious disease pathogen. Accordingly, the method of the present invention preferably increases a T cell response in an animal such that the animal is protected from a disease that is amenable to the stimulation of a memory T cell response, including cancer, an immunodeficiency disease and/or an infectious disease. As used herein, the phrase “protected from a disease” refers to reducing the symptoms of the disease; reducing the occurrence of the disease, and/or reducing the severity of the disease. Protecting an animal can refer to the ability of a therapeutic composition of the present invention, when administered to an animal, to prevent a disease from occurring and/or to cure or to alleviate disease symptoms, signs or causes. As such, to protect an animal from a disease includes both preventing disease occurrence (prophylactic treatment) and treating an animal that has a disease (therapeutic treatment). In particular, protecting an animal from a disease is accomplished by increasing a memory T cell response in the animal by increasing the proliferation and survival of memory T cells which, may, in some instances, additionally suppress (e.g., reduce, inhibit or block) an overactive or harmful immune response. The term, “disease” refers to any deviation from the normal health of an animal and includes a state when disease symptoms are present, as well as conditions in which a deviation (e.g., infection, gene mutation, genetic defect, etc.) has occurred, but symptoms are not yet manifested.

More specifically, a composition as described herein, when administered to an animal by the method of the present invention, preferably produces a result which can include alleviation of the disease, elimination of the disease, reduction of a tumor or lesion associated with the disease, elimination of a tumor or lesion associated with the disease, prevention of a secondary disease resulting from the occurrence of a primary disease (e.g., metastatic cancer resulting from a primary cancer), and prevention of the disease.

In an alternate embodiment of the present invention, a composition and method for decreasing an undesirable immune response, and particularly, for downregulating the activity of autoreactive T cells, as well as the activity of T cells recruited to the site of an autoimmune response (i.e., decreasing an autoimmune response), are described. For example, it is desirable to decrease (e.g., inhibit, downregulate, reduce) T cell memory responses in a patient that has an autoimmune disease (i.e., decrease memory T cell responses against an autoantigen), in a patient that has received an organ or cell transplant (i.e., decrease memory T cell responses against the organ or cell transplant), or in a patient that has an immunoproliferative disease (i.e., decrease memory T cell responses against a variety of antigens). In addition, it is desirable to increase the activity of CD25⁺ T cells and thereby suppress the activity of CD25⁻ T cells in a patient, by regulating the activity of IL-2 in a patient.

Preferably, the memory T cell response is decreased by administering to the patient a composition comprising at least one agent that decreases the activity of IL-15 in the patient and/or at least one agent that increases the activity of IL-2 in the patient. In a preferred embodiment, both agents are administered together in a composition with or without an antigen against which the memory T cell response is to be decreased. In a particularly preferred embodiment, the administration of the composition is targeted to a particular site or cell in a patient (e.g., a site of an autoimmune response, a transplanted organ), so that the effect of the composition is substantially localized to the T cells for which decreased response is desired.

Specifically, one aspect of this method of the present invention includes increasing the action of interleukin-2 (IL-2) in a patient wherein it is desirable to inhibit a particular memory T cell response (e.g., a patient that has or is at risk of developing an autoimmune T cell response). Reference to increasing the action (or activity) of IL-2 refers to any manipulation of the patient to be treated and specifically, of a cell of a patient to be treated, which results in increased functionality of IL-2 in the patient, including increasing the activity of IL-2 by acting on endogenous IL-2 or the receptor for IL-2, or by providing exogenous IL-2 or a compound that has IL-2 activity (e.g., by administration of exogenous IL-2 or a mimetic thereof, overexpression of IL-2 in the cells of the patient, administration of a compound that enhances endogenous IL-2 production, enhanced IL-2 receptor sensitivity or responsiveness in the cells of the patient, reduced inhibition of IL-2, and/or reduced degradation of IL-2).

In one embodiment, the action of IL-2 is increased in the patient by increasing the amount of IL-2 in the patient, and preferably at the site of an autoimmune response in a patient. The amount of IL-2 in the patient can be increased by any suitable method, including exogenous administration of IL-2 or mimetics thereof or by overexpressing IL-2 in the cells of a patient (e.g., by using recombinant technology or by administering compounds that enhance endogenous IL-2 expression in the cells of the patient). In a preferred embodiment, the action of IL-2 is increased in a manner that specifically targets the site of an autoimmune response in the patient. For example, exogenous IL-2 can be administered directly at the site of an autoimmune response, can be administered by ex vivo administration, or can be targeted to a particular cell or tissue type by linking the IL-2 to a targeting molecule and/or delivering the IL-2 in a targeting carrier. Targeting molecules can be any molecules suitable for delivering IL-2 or another compound of the present invention to a target site. Such molecules include, but are not limited to antibodies, ligands, soluble receptors or any protein or compound that is capable of selectively binding to a molecule on a target cell or tissue. Targeting molecules and suitable delivery vehicles are discussed in detail below. The site of an autoimmune response is the site, or location in the body of the patient (e.g., a cell, tissue, or general area of the body) wherein the autoimmune response is occurring. For example, type I diabetes mellitus is an autoimmune disease for which the site of the autoimmune response is the pancreas, and more specifically, the islet cells of the pancreas, and even more specifically, the beta cells in the islets. Any of these organs/cells can be generally targeted as the site of the autoimmune response in this example. Some autoimmune responses are systemic (e.g., systemic lupus erythematosus), and therefore the site of the autoimmune response is anywhere in which the T cells mediating the response can be targeted (e.g., by administration into the circulatory system). The appropriate site to administer a composition of the present invention will be apparent to those of skill in the art.

In one embodiment of the present invention, the action of IL-2 is increased in the patient in a manner effective to regulate the activity and/or survival of CD25⁺ T cells in the patient. Preferably, the activity of CD25⁺ T cells in the patient is enhanced, such that the CD25⁺ T cells survive for a longer period of time in the patient as compared to in the absence of increased IL-2 action and more particularly, such that the CD25⁺ T cells have an increased ability to suppress the responses of CD25⁻ T cells in the patient. In a preferred embodiment, the action of IL-2 is increased in the patient in a manner effective to regulate the activity of CD25⁺ T cells in the patient such that the activity of autoreactive CD25⁻ T cells is downregulated. More preferably, the activity of CD25⁻ T cells that are recruited to the site of an autoimmune response and that are associated with an autoimmune response are downregulated by the method of the present invention.

In one embodiment, the effects of IL-2 and of regulation of CD25⁺ T cells are directed to the site of an autoimmune response by administration of an appropriate dose of an autoantigen to the patient prior to, simultaneously with, or after administration of a composition or product of the present invention. It is noted, however, that the administration of antigen in connection with any of the products or methods of the present invention is not believed to be necessary to obtain the desired effect of regulation of T cell responses and particularly, of regulation of CD25⁺ T cells.

In a further embodiment of the present invention, the method of downregulating a T cell response includes the step of downregulating the action (i.e., activity) of interleukin-15 (IL-15) in the patient, alone or preferably, in addition to, increasing the action of IL-2. Reference to downregulating or decreasing the action (or activity) of IL-15 refers to any manipulation of the patient to be treated and specifically, of a cell of a patient to be treated, which results in decreased functionality of IL-15 in the patient, including decreased activity of IL-15 by acting on endogenous IL-15 or the receptor for IL-15 (e.g., by administration of an antibody that specifically binds to and blocks the activity of IL-15 or results in elimination of IL-15; by administration of an antibody that specifically prevents the interaction of IL-15 with its receptor, preferably without inhibiting the interaction of IL-2 with its receptor; by administering a compound that decreases endogenous IL-15 production; by administering a compound that decreases IL-15 receptor sensitivity or responsiveness in the cells of the patient without decreasing IL-2 receptor responsiveness in a patient; or increasing degradation of IL-15 in the patient).

Autoimmune diseases to treat using the method of the present invention include any autoimmune disease which is associated with an autoreactive T cell response, including, but not limited to, rheumatoid arthritis, systemic lupus erythematosus, insulin dependent diabetes mellitis, multiple sclerosis, myasthenia gravis, and Grave's disease.

Downregulating a T cell response in an animal can be an effective treatment for a wide variety of medical disorders, and in particular, for autoimmune disease and/or graft rejection. As used herein, the term “downregulate” can be used interchangeably with the terms “decrease”, “inhibit”, or “reduce”. According to the present invention, “downregulating a T cell response” in an animal refers to specifically controlling or influencing the activity of the T cell response, and can include preventing the T cell response, decreasing the T cell response, and/or inhibiting the T cell response, and more particularly, a memory T cell response.

Preferably, the method of the present invention decreases a memory T cell response and/or increases CD25⁺ T cell responses at the site of an autoimmune response or other undesirable immune response in a patient. Accordingly, the method of the present invention preferably decreases a memory T cell response or increases a CD25⁺ T cell response in an animal such that the animal is protected from an autoimmune disease or other disease/conditions mediated by an undesirable immune response that is amenable to the inhibition of a memory T cell response and/or stimulation of a CD25⁺ T cell response. More specifically, a composition as described herein, when administered to an animal by the method of the present invention, preferably produces a result which can include alleviation of the disease, elimination of the disease, prevention of disease recurrence, and prevention of the disease.

As used herein, the biological activity or biological action of a protein (i.e., the activity) refers to any function(s) exhibited or performed by a naturally occurring form of the protein as measured or observed in vivo (i.e., in the natural physiological environment of the protein) or in vitro (i.e., under laboratory conditions). The biological activity of IL-2 or IL-15 and homologues thereof can be evaluated, for example, by measuring the ability of the cytokine or homologue thereof to bind to and activate the receptor for the cytokine, or to act on cells known to be supported by either cytokine in an in vitro assay (e.g., a T cell proliferation assay). For example, a biological activity of IL-2 can include, but is not limited to, support of proliferation or induction of apoptosis of activated T cells, generation of CTL activity, stimulation of B cell growth and J-chain synthesis, stimulation of NK cell growth, and as demonstrated or suggested herein, the ability to kill proliferating CD8+ memory phenotype cells and/or sustain the growth of CD25⁺ regulatory T cells. Biological activities of IL-15 can include, but are not limited to, the ability to support the proliferation of a T cell (which can be measured, for example, by the ability of IL-15 to support proliferation of a T cell line in vitro), the ability to induce immunoglobulin secretion from activated B cells, and as demonstrated herein, the ability to support the growth and proliferation of memory T cells. Modifications of a protein, such as in a homologue or mimetic (discussed below), which result in a decrease in protein expression or a decrease in the activity of the protein, can be referred to as inactivation (complete or partial), down-regulation, or decreased action of a protein. Similarly, modifications which result in an increase in protein expression or an increase in the activity of the protein, can be referred to as amplification, overproduction, activation, enhancement, up-regulation or increased action of a protein. IL-2 or IL-15 biological activity can be evaluated by one of skill in the art by any suitable in vitro or in vivo assay for measuring cytokine binding to its receptor, or cytokine activity.

Accordingly, the methods of the present invention include the use of a variety of agents (i.e., regulatory compounds) which, by acting directly on IL-2 or IL-15, their receptors, or the genes encoding IL-2, IL-15 or their receptors, increase or decrease the activity of IL-2 or IL-15 in a patient such that the desired result is achieved (e.g., enhancement of memory T cell responses or inhibition of autoimmune responses). Agents useful in the present invention include, for example, proteins, nucleic acid molecules, antibodies, and compounds that are products of rational drug design (i.e., drugs). More specifically, such agents include, but are not limited to, the cytokines (IL-2 or IL-15), biologically active portions thereof, or homologues or mimetics thereof; nucleic acid molecules that encode the cytokines; antibodies that bind to the cytokines or to the receptor for the cytokines (including stimulatory and blocking or neutralizing antibodies); antisense nucleic acids that hybridize to the genes encoding the cytokines and inhibit transcription of the gene; a protein or nucleic acid sequence that binds to a regulatory region of the genes encoding the cytokines and stimulates transcription of the gene; or a small molecule (e.g., a product of drug design) that agonizes or antagonizes the action of the cytokine or its receptor).

One type of agent that is useful for regulating the activity of IL-2 and/or IL-15 includes an antibody that selectively binds to IL-2 or IL-15 or to a receptor for IL-2 or IL-15 (i.e., IL-2R or IL-15R, respectively). The antibody can be a stimulating antibody, a blocking antibody or a neutralizing antibody, depending on the action that is desired of the agent (e.g., if increase of IL-2 activity is desired, then the antibody can be a stimulating antibody that selectively binds to and stimulates the IL-2R; if inhibition of IL-15 activity is desired, then the antibody can be a blocking antibody that selectively binds to IL-15 or IL-15R). Preferably, if the antibody selectively binds to a receptor for IL-2 or IL-15, it does not bind to a receptor chain that is shared by the IL-2-receptor and the IL-15-receptor, so that the antibody selectively inhibits the binding of one cytokine to its receptor, but not the other, or so that the antibody stimulates one receptor, but not the other. In a preferred embodiment, an antibody that selectively binds to IL-2R but not to IL-15R is an antibody that selectively binds to the IL-2Rα chain, also known as CD25. In this embodiment, when it is desirable to inhibit the activity of IL-2, but not IL-15, a blocking antibody that selectively binds to IL-2Rα can be used. Such an antibody does not bind to the receptor for IL-15, and therefore the action of IL-15 is not inhibited. Similarly, when it is desirable to inhibit the activity of IL-15, but not IL-2, one can use a blocking antibody that selectively binds to the IL-15Rα chain. The IL-2R and the IL-15R share the IL-2Rβ and γc chains, but antibodies that bind to IL-2Rβ and block IL-15, but not IL-2, for example, are known in the art (e.g., de Jong et al., 1998, Cytokine 10(12):920-930) and could be used in the present invention. Antibodies that block or stimulate both the IL-2R and the IL-15R are not preferred for use in the present invention.

According to the present invention, the phrase “selectively binds to” refers to the ability of an antibody, antigen binding fragment or binding partner used in the present invention to preferentially bind to specified proteins (e.g., to IL-2, IL-2R, IL-15 or IL-15R). More specifically, the phrase “selectively binds” refers to the specific binding of one protein to another (e.g., an antibody, fragment thereof, or binding partner to an antigen), wherein the level of binding, as measured by any standard assay (e.g., an immunoassay), is statistically significantly higher than the background control for the assay. For example, when performing an immunoassay, controls typically include a reaction well/tube that contain antibody or antigen binding fragment alone (i.e., in the absence of antigen), wherein an amount of reactivity (e.g., non-specific binding to the well) by the antibody or antigen binding fragment thereof in the absence of the antigen bound by the antibody is considered to be background. Binding can be measured using a variety of methods standard in the art including enzyme immunoassays (e.g., ELISA), immunoblot assays, etc.

According to the present invention, an antigen binding fragment can include an Fab, an Fab′, or an F(ab′)₂ fragment of an immunoglobulin. A fragment lacking the ability to bind to antigen is referred to as an Fc fragment. An Fab fragment comprises one arm of an immunoglobulin molecule containing a L chain (V_{L}+C_{L} domains) paired with the V_{H} region and a portion of the C_{H} region (CH1 domain). An Fab′ fragment corresponds to an Fab fragment with part of the hinge region attached to the CH1 domain. An F(ab′)₂ fragment corresponds to two Fab′ fragments that are normally covalently linked to each other through a di-sulfide bond, typically in the hinge regions.

Functional aspects of an immunoglobulin molecule include the valency of an immunoglobulin molecule, the affinity of an immunoglobulin molecule, and the avidity of an immunoglobulin molecule. As used herein, affinity refers to the strength with which an immunoglobulin molecule binds to an antigen at a single site on an immunoglobulin molecule (i.e., a monovalent Fab fragment binding to a monovalent antigen). Affinity differs from avidity which refers to the sum total of the strength with which an immunoglobulin binds to an antigen. Immunoglobulin binding affinity can be measured using techniques standard in the art, such as competitive binding techniques, equilibrium dialysis or BlAcore methods. As used herein, valency refers to the number of different antigen binding sites per immunoglobulin molecule (i.e., the number of antigen binding sites per antibody molecule of antigen binding fragment). For example, a monovalent immunoglobulin molecule can only bind to one antigen at one time, whereas a bivalent immunoglobulin molecule can bind to two or more antigens at one time, and so forth. Both monovalent and bivalent antibodies that selectively bind to a protein (e.g., IL-2) in a manner useful in the present invention are encompassed herein.

In one embodiment, the antibody is a bi- or multi-specific antibody. A bi-specific (or multi-specific) antibody is capable of binding two (or more) antigens, as with a divalent (or multivalent) antibody, but in this case, the antigens are different antigens (i.e., the antibody exhibits dual or greater specificity). A bi-specific antibody suitable for use in the present method includes an antibody having: (a) a first portion (e.g., a first antigen binding portion) which binds to a first antigen (e.g., an IL-2Rα chain (CD25) of an IL-2R); and (b) a second portion which binds to a second antigen (e.g., a cell surface molecule expressed by a cell which expresses the IL-2R). In this example, the second portion can bind to any cell surface molecule. In a preferred embodiment, the second portion is capable of targeting the antibody to a specific target cell in which or near which it is desirable to increase or decrease the activity of IL-2 or IL-15 (i.e., the regulatory antibody binds to a target molecule). Monovalent or divalent antibodies can also be linked to an agent for increasing or decreasing the action of IL-2 or IL-15 and used as targeting moieties.

Isolated antibodies of the present invention can include serum containing such antibodies, or antibodies that have been purified to varying degrees. Whole antibodies of the present invention can be polyclonal or monoclonal. Alternatively, functional equivalents of whole antibodies, such as antigen binding fragments in which one or more antibody domains are truncated or absent (e.g., Fv, Fab, Fab′, or F(ab)₂ fragments), as well as genetically-engineered antibodies or antigen binding fragments thereof, including single chain antibodies or antibodies that can bind to more than one epitope (e.g., bi-specific antibodies), or antibodies that can bind to one or more different antigens (e.g., bi- or multi-specific antibodies), may also be employed in the invention.

Genetically engineered antibodies of the invention include those produced by standard recombinant DNA techniques involving the manipulation and re-expression of DNA encoding antibody variable and/or constant regions. Particular examples include, chimeric antibodies, where the V_{H} and/or V_{L} domains of the antibody come from a different source to the remainder of the antibody, and CDR grafted antibodies (and antigen binding fragments thereof), in which at least one CDR sequence and optionally at least one variable region framework amino acid is (are) derived from one source and the remaining portions of the variable and the constant regions (as appropriate) are derived from a different source. Construction of chimeric and CDR-grafted antibodies are described, for example, in European Patent Applications: EP-A 0194276, EP-A 0239400, EP-A 0451216 and EP-A 0460617.

Alternative methods, employing, for example, phage display technology (see for example U.S. Pat. No. 5,969,108, U.S. Pat. No. 5,565,332, U.S. Pat. No. 5871907, U.S. Pat. No. 5,858,657) or the selected lymphocyte antibody method of U.S. Pat. No. 5,627,052 may also be used for the production of antibodies and/or antigen fragments of the invention, as will be readily apparent to the skilled individual.

Generally, in the production of an antibody, a suitable experimental animal, such as, for example, but not limited to, a rabbit, a sheep, a hamster, a guinea pig, a mouse, a rat, or a chicken, is exposed to an antigen against which an antibody is desired. Typically, an animal is immunized with an effective amount of antigen that is injected into the animal. An effective amount of antigen refers to an amount needed to induce antibody production by the animal. The animal's immune system is then allowed to respond over a pre-determined period of time. The immunization process can be repeated until the immune system is found to be producing antibodies to the antigen. In order to obtain polyclonal antibodies specific for the antigen, serum is collected from the animal that contains the desired antibodies (or in the case of a chicken, antibody can be collected from the eggs). Such serum is useful as a reagent. Polyclonal antibodies can be further purified from the serum (or eggs) by, for example, treating the serum with ammonium sulfate.

Monoclonal antibodies maybe produced according to the methodology of Kohler and Milstein (Nature 256:495-497, 1975). For example, B lymphocytes are recovered from the spleen (or any suitable tissue) of an immunized animal and then fused with myeloma cells to obtain a population of hybridoma cells capable of continual growth in suitable culture medium. Hybridomas producing the desired antibody are selected by testing the ability of the antibody produced by the hybridoma to bind to the desired antigen.

Antibodies that selectively bind to IL-2, IL-15, IL-2R or IL-15R are known in the art. For example, antibodies against human and mouse IL-2, human IL-15, and human and mouse IL-2Rα (CD25) are commercially available from PharMingen, San Diego, Calif. Antibodies against IL-2, IL-2Rβ and IL-2Rα (CD25) are described in the Examples.

Another class of agents useful in the methods of the present invention are the cytokines, IL-2 and IL-15, biologically active fragments thereof, and homologues or mimetics thereof. Generally, such agents are useful for increasing the activity of IL-2 or IL-15, although some homologues or mimetics may actually be designed or selected to be antagonists of IL-2 or IL-15 and are therefore useful for decreasing the activity of IL-2 or IL-15. According to the present invention, a protein having “biological activity” or a “biologically active fragment” of a protein, such as a protein having IL-2 biological activity (or IL-2 activity) can be a full-length protein or any homologue of such a protein (e.g., a protein in which amino acids have been deleted (e.g., a truncated version of the protein, such as a biologically active peptide or fragment), inserted, inverted, substituted and/or derivatized (e.g., by glycosylation, phosphorylation, acetylation, myristoylation, prenylation, palmitation, amidation and/or addition of glycosylphosphatidyl inositol)). Biological activity of IL-2 and IL-15 has been described in detail above. A homologue of a given protein is a protein having an amino acid sequence that is sufficiently similar to a naturally occurring protein amino acid sequence that the homologue has substantially the same or enhanced biological activity compared to the corresponding naturally occurring protein. The functional domains of a wild-type IL-2 protein and a wild-type IL-15 protein are known in the art, and therefore, one of skill in the art would be able to selectively modify a wild-type IL-2 or IL-15 as discussed above to develop a homologue of the cytokine with substantially similar biological activity to the natural cytokine. Moreover, the nucleic acid sequence and amino acid sequence for these cytokines in several mammalian species are known in the art. For example, the nucleic acid and amino acid sequences for human IL-2 are published in GenBank as Accession Nos. AF359939 (gene) and AAK26665 (protein); the nucleic acid and amino acid sequences for human IL-15 are published in GenBank as Accession Nos. X91233 (gene) and CAA62616.1 (protein); the nucleic acid and amino acid sequences for mouse IL-2 are published in GenBank as Accession Nos. AF195956 (gene) and AAF32272.1 (protein); and the nucleic acid and amino acid sequences for mouse IL-15 are published in GenBank as Accession Nos. U14332 (mRNA), AB006745 (promoter and partial gene), and AAA75377 (protein).

More particularly, a homologue of an IL-2 or IL-15 protein has an amino acid sequence that is at least about 70% identical to the amino acid sequence of a naturally occurring IL-2 or IL-15 protein, respectively, and more preferably, at least about 75%, and more preferably, at least about 80%, and more preferably, at least about 85%, and more preferably, at least about 90%, and more preferably, at least about 95% identical to the amino acid sequence of a naturally occurring IL-2 or IL-15, respectively. As used herein, unless otherwise specified, reference to a percent (%) identity refers to an evaluation of homology which is performed using a BLAST homology search. BLAST homology searches can be performed using the BLAST database and software, which offers search programs including: (1) a BLAST 2.0 Basic BLAST homology search (http://www.ncbi.nlm.nih.gov/BLAST) using blastp for amino acid searches and blastn for nucleic acid searches with standard default parameters, wherein the query sequence is filtered for low complexity regions by default (described in Altschul, S. F., Madden, T. L., Sch\ddot{aa}ffer, A. A., Zhang, J., Zhang, Z., Miller, W. & Lipman, D. J. (1997) “Gapped BLAST and PSI-BLAST: a new generation of protein database search programs.” Nucleic Acids Res. 25:3389-3402, incorporated herein by reference in its entirety); (2) a BLAST 2 alignment (using the parameters described below) ((http://www.ncbi.nlm.nih.gov/BLAST); (3) and/or PSI-BLAST with the standard default parameters (Position-Specific Iterated BLAST; (http://www.ncbi.nlm.nih.gov/BLAST). It is noted that due to some differences in the standard parameters between BLAST 2.0 Basic BLAST and BLAST 2, two specific sequences might be recognized as having significant homology using the BLAST 2 program, whereas a search performed in BLAST 2.0 Basic BLAST using one of the sequences as the query sequence may not identify the second sequence in the top matches.

Two specific sequences can be aligned to one another using BLAST 2 sequence as described in Tatusova and Madden, (1999), “Blast 2 sequences—a new tool for comparing protein and nucleotide sequences”, FEMS Microbiol Lett. 174:247-250, incorporated herein by reference in its entirety. BLAST 2 sequence alignment is performed in blastp or blastn using the BLAST 2.0 algorithm to perform a Gapped BLAST search (BLAST 2.0) between the two sequences allowing for the introduction of gaps (deletions and insertions) in the resulting alignment. For purposes of clarity herein, a BLAST 2 sequence alignment is performed using the standard default parameters as follows.

For blastn, using 0 BLOSUM62 matrix:

Reward for match=1

Penalty for mismatch=−2

Open gap (5) and extension gap (2) penalties

gap x_dropoff (50) expect (10) word size (11) filter (on)

For blastp, using 0 BLOSUM62 matrix:

Open gap (11) and extension gap (1) penalties

gap x_dropoff (50) expect (10) word size (3) filter (on).

In addition, PSI-BLAST provides an automated, easy-to-use version of a “profile” search, which is a sensitive way to look for sequence homologues. The program first performs a gapped BLAST database search. The PSI-BLAST program uses the information from any significant alignments returned to construct a position-specific score matrix, which replaces the query sequence for the next round of database searching. Therefore, it is to be understood that percent identity can be determined by using any one of these programs, although for the direct comparison of two sequences, BLAST 2 is preferred.

In another embodiment, an IL-2 or IL-15 homologue of the present invention includes a protein having an amino acid sequence that is sufficiently similar to the naturally occurring IL-2 or IL-15 amino acid sequence, respectively, that a nucleic acid sequence encoding the homologue is capable of hybridizing under high stringency conditions (described below) to (i.e., with) a nucleic acid molecule encoding the naturally occurring IL-2 or IL-15 protein, respectively (i.e., to the complement of the nucleic acid strand encoding the naturally occurring cytokine amino acid sequence).

Preferred proteins having IL-2 or IL-15 biological activity which can be administered to a patient and/or expressed in a cell (i.e., when delivered by a recombinant nucleic acid molecule encoding the protein) according to the method of the present invention include, but are not limited to any isolated, synthetically produced and/or recombinantly produced wild-type (e.g., naturally occurring) IL-2 or IL-15, as well as homologues of such proteins. According to the present invention, a wild-type IL-2 or IL-15 is an IL-2 or IL-15 protein that can be isolated from any species of the kingdom, Animalia, and which is characterized by its ability to bind to a receptor for the cytokine which results in stimulation or an increase in the activity of the receptor. Homologues have been described above.

An IL-2 or IL-15 protein useful in the present methods can also include a fusion protein, that includes a cytokine protein-containing domain (i.e., IL-2- or IL-15-containing domain) attached to one or more fusion segments. Suitable fusion segments for use with the present invention include, but are not limited to, any segments that can enhance the biological activity of the cytokine, facilitate the purification of the fusion protein from a production cell, or enhance the protein's stability in the host cell (e.g., increase the half-life of the cytokine). A suitable fusion segment can be a domain of any size that has the desired function.

Another agent useful in the methods of the present invention includes a mimetic of IL-2 or IL-15. As used herein, the term “mimetic” is used to refer to any peptide or non-peptide compound that is able to mimic the biological action of a naturally occurring peptide, often because the mimetic has a basic structure that mimics the basic structure of the naturally occurring peptide and/or has the salient biological properties of the naturally occurring peptide. Mimetics can also be designed which antagonize the biological activity of a naturally occurring peptide. Mimetics can include, but are not limited to: peptides that have substantial modifications from the prototype such as no side chain similarity with the naturally occurring peptide (such modifications, for example, may decrease its susceptibility to degradation); anti-idiotypic and/or catalytic antibodies, or fragments thereof; non-proteinaceous portions of an isolated protein (e.g., carbohydrate structures); or synthetic or natural organic molecules, including nucleic acids and drugs identified through combinatorial chemistry, for example. Such mimetics can be designed, selected and/or otherwise identified using a variety of methods known in the art.

Various methods of drug design, useful to design mimetics or other therapeutic compounds useful in the present invention are disclosed in Maulik et al., 1997, Molecular Biotechnology: Therapeutic Applications and Strategies, Wiley-Liss, Inc., which is incorporated herein by reference in its entirety. An IL-2 or IL-15 agonist or antagonist can be obtained, for example, from molecular diversity strategies (a combination of related strategies allowing the rapid construction of large, chemically diverse molecule libraries), libraries of natural or synthetic compounds, in particular from chemical or combinatorial libraries (i.e., libraries of compounds that differ in sequence or size but that have the similar building blocks) or by rational, directed or random drug design. See for example, Maulik et al., supra.

In a molecular diversity strategy, large compound libraries are synthesized, for example, from peptides, oligonucleotides, carbohydrates and/or synthetic organic molecules, using biological, enzymatic and/or chemical approaches. The critical parameters in developing a molecular diversity strategy include subunit diversity, molecular size, and library diversity. The general goal of screening such libraries is to utilize sequential application of combinatorial selection to obtain high-affinity ligands for a desired target, and then to optimize the lead molecules by either random or directed design strategies. Methods of molecular diversity are described in detail in Maulik, et al., ibid.

Maulik et al. also disclose, for example, methods of directed design, in which the user directs the process of creating novel molecules from a fragment library of appropriately selected fragments; random design, in which the user uses a genetic or other algorithm to randomly mutate fragments and their combinations while simultaneously applying a selection criterion to evaluate the fitness of candidate ligands; and a grid-based approach in which the user calculates the interaction energy between three dimensional receptor structures and small fragment probes, followed by linking together of favorable probe sites.

In one aspect of the present invention, an agent that is useful for increasing the activity of IL-2 or IL-15 to the present invention, is a nucleic acid molecule encoding IL-2 or IL-15, or a homologue thereof. Such a nucleic acid molecule is intended to be delivered to and expressed by a cell in the patient, thereby increasing the activity of IL-2 or IL-15 in the patient. The nucleic acid sequence is typically included in a recombinant nucleic acid molecule. A recombinant nucleic acid molecule of the present invention is a molecule that can include at least one of any nucleic acid sequence encoding a protein having IL-2 or IL-15 biological activity operatively linked to at least one of any transcription control sequence capable of effectively regulating expression of the nucleic acid molecule(s) in the cell to be transfected. Although the phrase “nucleic acid molecule” primarily refers to the physical nucleic acid molecule and the phrase “nucleic acid sequence” primarily refers to the sequence of nucleotides on the nucleic acid molecule, the two phrases can be used interchangeably, especially with respect to a nucleic acid molecule, or a nucleic acid sequence, being capable of encoding a protein. In addition, the phrase “recombinant molecule” primarily refers to a nucleic acid molecule operatively linked to a transcription control sequence, but can be used interchangeably with the phrase “nucleic acid molecule” which can be administered to an animal.

In accordance with the present invention, an isolated nucleic acid molecule is a nucleic acid molecule that has been removed from its natural milieu (i.e., that has been subject to human manipulation). As such, “isolated” does not reflect the extent to which the nucleic acid molecule has been purified. An isolated nucleic acid molecule can include DNA, RNA, or derivatives of either DNA or RNA. There is no limit, other than a practical limit, on the maximal size of a nucleic acid molecule in that the nucleic acid molecule can include a portion of a gene, an entire gene, multiple genes, or portions thereof.

An isolated nucleic acid molecule of the present invention can be obtained from its natural source either as an entire (i.e., complete) gene or a portion thereof capable of forming a stable hybrid with that gene. Preferably, an isolated nucleic acid molecule is produced using recombinant DNA technology (e.g., polymerase chain reaction (PCR) amplification, cloning) or chemical synthesis. Isolated nucleic acid molecules include natural nucleic acid molecules and homologues thereof, including, but not limited to, natural allelic variants and modified nucleic acid molecules in which nucleotides have been inserted, deleted, substituted, and/or inverted, but wherein the modifications do not substantially decrease the activity encoded protein as compared to the naturally occurring protein.

A nucleic acid molecule homologue can be produced using a number of methods known to those skilled in the art (see, for example, Sambrook et al., ibid.). For example, nucleic acid molecules can be modified using a variety of techniques including, but not limited to, classic mutagenesis techniques and recombinant DNA techniques, such as site-directed mutagenesis, chemical treatment of a nucleic acid molecule to induce mutations, restriction enzyme cleavage of a nucleic acid fragment, ligation of nucleic acid fragments, PCR amplification and/or mutagenesis of selected regions of a nucleic acid sequence, synthesis of oligonucleotide mixtures and ligation of mixture groups to “build” a mixture of nucleic acid molecules and combinations thereof. Nucleic acid molecule homologues can be selected from a mixture of modified nucleic acids by screening for the function of the protein encoded by the nucleic acid and/or by hybridization with a wild-type gene. Preferred nucleic acid molecules according to the present invention are any isolated nucleic acid molecules which comprise a nucleic acid sequence encoding an IL-2 or IL-15 protein having IL-2 or IL-15 biological activity, respectively, as described above.

Knowing the nucleic acid sequences of certain nucleic acid molecules of the present invention allows one skilled in the art to, for example, (a) make copies of those nucleic acid molecules and/or (b) obtain nucleic acid molecules including at least a portion of such nucleic acid molecules (e.g., nucleic acid molecules including full-length genes, full-length coding regions, regulatory control sequences, truncated coding regions). Such nucleic acid molecules can be obtained in a variety of ways including traditional cloning techniques using oligonucleotide probes to screen appropriate libraries or DNA and PCR amplification of appropriate libraries or DNA using oligonucleotide primers. Preferred libraries to screen or from which to amplify nucleic acid molecule include mammalian genomic DNA libraries. Techniques to clone and amplify genes are disclosed, for example, in Sambrook et al., ibid.

A recombinant nucleic acid molecule includes a recombinant vector, which is any nucleic acid sequence, typically a heterologous sequence, which is operatively linked to the isolated nucleic acid molecule encoding the IL-2 or IL-15 protein, which is capable of enabling recombinant production of the protein, and which is capable of delivering the nucleic acid molecule into a host cell according to the present invention. Such a vector can contain nucleic acid sequences that are not naturally found adjacent to the isolated nucleic acid molecules to be inserted into the vector. The vector can be either RNA or DNA, either prokaryotic or eukaryotic, and preferably in the present invention, is a virus or a plasmid. Recombinant vectors can be used in the cloning, sequencing, and/or otherwise manipulating of nucleic acid molecules. Recombinant vectors are preferably used in the expression of nucleic acid molecules, and can also be referred to as expression vectors. Preferred recombinant vectors are capable of being expressed in a transfected host cell, and particularly, in a transfected mammalian host cell ex vivo or in vivo.

In a recombinant molecule, nucleic acid molecules are operatively linked to expression vectors containing regulatory sequences such as transcription control sequences, translation control sequences, origins of replication, and other regulatory sequences that are compatible with the host cell and that control the expression of nucleic acid molecules of the present invention. In particular, recombinant molecules of the present invention include nucleic acid molecules that are operatively linked to one or more transcription control sequences. The phrase “operatively linked” refers to linking a nucleic acid molecule to a transcription control sequence in a manner such that the molecule is able to be expressed when transfected (i.e., transformed, transduced or transfected) into a host cell.

Transcription control sequences are sequences which control the initiation, elongation, and termination of transcription. Particularly important transcription control sequences are those which control transcription initiation, such as promoter, enhancer, operator and repressor sequences. Suitable transcription control sequences include any transcription control sequence that can function in a host cell according to the present invention. A variety of suitable transcription control sequences are known to those skilled in the art. Preferred transcription control sequences include those which function in mammalian cells, with cell- or tissue-specific transcription control sequences being particularly preferred. Particularly preferred transcription control sequences include inducible promoters, cell-specific promoters, tissue-specific promoters (e.g., insulin promoters) and enhancers. Suitable promoters for these and other cell types will be easily determined by those of skill in the art. Transcription control sequences of the present invention can also include naturally occurring transcription control sequences naturally associated with the protein to be expressed prior to isolation. In one embodiment, a transcription control sequence includes an inducible promoter.

Recombinant molecules of the present invention may also contain fusion sequences which lead to the expression of nucleic acid molecules as fusion proteins. Eukaryotic recombinant molecules may include intervening and/or untranslated sequences surrounding and/or within the nucleic acid sequences of nucleic acid molecules.

One type of recombinant vector useful in a recombinant nucleic acid molecule of the present invention is a recombinant viral vector. Such a vector includes a recombinant nucleic acid sequence encoding an IL-2 or IL-15 protein that is packaged in a viral coat that can be expressed in a host cell in an animal or ex vivo after administration. A number of recombinant viral vectors can be used, including, but not limited to, those based on alphaviruses, poxviruses, adenoviruses, herpesviruses, lentiviruses, adeno-associated viruses and retroviruses. Particularly preferred viral vectors are those based on adenoviruses and adeno-associated viruses. Viral vectors suitable for gene delivery are well known in the art and can be selected by the skilled artisan for use in the present invention. A detailed discussion of current viral vectors is provided in “Molecular Biotechnology,” Second Edition, by Glick and Pasternak, ASM Press, Washington D.C., 1998, pp. 555-590, the entirety of which is incorporated herein by reference.

For example, a retroviral vector, which is useful when it is desired to have a nucleic acid sequence inserted into the host genome for long term expression, can be packaged in the envelope protein of another virus so that it has the binding specificity and infection spectrum that are determined by the envelope protein (e.g., a pseudotyped virus). In addition, the envelope gene can be genetically engineered to include a DNA element that encodes and amino acid sequence that binds to a cell receptor to create a recombinant retrovirus that infects a specific cell type. Expression of the gene (e.g., the IL-2 gene) can be further controlled by the use of a cell or tissue-specific promoter. Retroviral vectors have been successfully used to transfect cells with a gene which is expressed and maintained in a variety of ex vivo systems

An adenoviral vector is a preferred vector for use in the present method. An adenoviral vector infects a wide range of nondividing human cells and has been used extensively in live vaccines without adverse side effects. Adenoviral vectors do not integrate into the host genome, and therefore, gene therapy using this system requires periodic administration, although methods have been described which extend the expression time of adenoviral transferred genes, such as administration of antibodies directed against T cell receptors at the site of expression (Sawchuk et al., 1996, Hum. Gene. Ther. 7:499-506), although this method is not preferred in the present invention. The efficiency of adenovirus-mediated gene delivery can be enhanced by developing a virus that preferentially infects a particular target cell. For example, a gene for the attachment fibers of adenovirus can be engineered to include a DNA element that encodes a protein domain that binds to a cell-specific receptor.

Yet another type of viral vector is based on adeno-associated viruses, which are small, nonpathogenic, single-stranded human viruses. This virus can integrate into a specific site on chromosome 19. This virus can carry a cloned insert of about 4.5 kb, and has typically been successfully used to express proteins in vivo from 70 days to at least 5 months. Demonstrating that the art is quickly advancing in the area of gene therapy, however, a recent publication by Bennett et al. reported efficient and stable transgene expression by adeno-associated viral vector transfer in vivo for greater than 1 year (Bennett et al., 1999, Proc. Natl. Acad. Sci. USA 96:9920-9925).

Another type of viral vector that is suitable for use in the present invention is a herpes simplex virus vector. Herpes simplex virus type 1 infects and persists within nondividing neuronal cells, and is therefore a suitable vector for targeting and transfecting cells of the central and peripheral nervous system with an IL-2 or IL-15 protein of the present invention. Preclinical trials in experimental animal models with such a vector has demonstrated that the vector can deliver genes to cells of both the brain and peripheral nervous system that are expressed and maintained for long periods of time.

One or more recombinant molecules of the present invention can be used to produce an encoded product (i.e., a protein having biological activity) useful in the method of the present invention. In one embodiment, an encoded product is produced by expressing a recombinant nucleic acid molecule as described herein under conditions effective to produce the protein. A preferred method to produce an encoded protein is by transfecting a host cell (i.e., a target cell) with one or more recombinant molecules to form a recombinant cell. Suitable host cells to transfect include any mammalian cell that can be transfected. Host cells can be either untransfected cells or cells that are already transfected with at least one nucleic acid molecule. Host cells according to the present invention can be any cell capable of producing a protein as described herein. A preferred host cell includes any mammalian cell, and more preferably, cells at the site of a condition to be treated using the present method (e.g., the site of a tumor, a vaccination site, the site of an autoimmune response).

As used herein, the term “target cell” refers to a cell to which a composition of the present invention is selectively designed to be delivered. The term target cell does not necessarily restrict the delivery of a recombinant nucleic acid molecule only to the target cell and no other cell, but indicates that the delivery of the recombinant molecule, the expression of the recombinant molecule, or both, are specifically directed to a preselected host cell. Targeting delivery vehicles, including liposomes and viral vector systems are known in the art. For example, a liposome can be directed to a particular target cell or tissue by using a targeting agent, such as an antibody, soluble receptor or ligand, incorporated with the liposome, to target a particular cell or tissue to which the targeting molecule can bind. Targeting liposomes are described, for example, in Ho et al., 1986, Biochemistry 25: 5500-6; Ho et al., 1987a, J Biol Chem 262: 13979-84; Ho et al., 1987b, J Biol Chem 262: 13973-8; and U.S. Pat. No. 4,957,735 to Huang et al., each of which is incorporated herein by reference in its entirety). Ways in which viral vectors can be modified to deliver a nucleic acid molecule to a target cell have been discussed above. Alternatively, the route of administration, as discussed below, can be used to target a specific cell or tissue. For example, intracoronary administration of an adenoviral vector has been shown to be effective for the delivery of a gene cardiac myocytes (Maurice et al., 1999, J. Clin. Invest. 104:21-29). Intravenous delivery of cholesterol-containing cationic liposomes has been shown to preferentially target pulmonary tissues (Liu et al., Nature Biotechnology 15:167, 1997), and effectively mediate transfer and expression of genes in vivo. Finally, a recombinant nucleic acid molecule can be selectively (i.e., preferentially, substantially exclusively) expressed in a target cell by selecting a transcription control sequence, and preferably, a promoter, which is selectively induced in the target cell and remains substantially inactive in non-target cells.

An isolated nucleic acid molecule that is particularly useful as an agent for inhibiting the activity of IL-2 or IL-15 is an anti-sense nucleic acid molecule. As used herein, an anti-sense nucleic acid molecule is defined as an isolated nucleic acid molecule that reduces expression of IL-2 or IL-15 by hybridizing under high stringency conditions to a gene encoding IL-2 or IL-15, respectively. Such a nucleic acid molecule is sufficiently similar to the nucleic acid sequence encoding the IL-2 or IL-15 that the molecule is capable of hybridizing under high stringency conditions to the coding strand of the gene or RNA encoding the natural protein. An IL-2 gene or an IL-15 gene includes all nucleic acid sequences related to the gene such as regulatory regions that control production of the protein encoded by that gene (such as, but not limited to, transcription, translation or post-translation control regions) as well as the coding region itself. As discussed above, the genes encoding IL-2 and IL-15 have been previously cloned and sequenced and are available to those of skill in the art. Preferably, an anti-sense molecule of the present invention is at least about 25 nucleotides in length, and more preferably at least about 50 nucleotides in length, and more preferably at least about 75 nucleotides in length, and more preferably at least about 100 nucleotides in length, and more preferably at least about 125 nucleotides in length, and more preferably at least about 150 nucleotides in length, and more preferably at least about 200 nucleotides in length, and more preferably at least about 250 nucleotides in length, and more preferably at least about 300 nucleotides in length, and more preferably at least about 350 nucleotides in length, and more preferably at least about 400 nucleotides in length.

As used herein, stringent hybridization conditions refer to standard hybridization conditions under which nucleic acid molecules are used to identify similar nucleic acid molecules. Such standard conditions are disclosed, for example, in Sambrook et al., Molecular Cloning: A Laboratory Manual, Cold Spring Harbor Labs Press, 1989. Sambrook et al., ibid., is incorporated by reference herein in its entirety (see specifically, pages 9.31-9.62, 11.7 and 11.45-11.61). In addition, formulae to calculate the appropriate hybridization and wash conditions to achieve hybridization permitting varying degrees of mismatch of nucleotides are disclosed, for example, in Meinkoth et al., 1984, Anal. Biochem. 138, 267-284; Meinkoth et al., ibid., is incorporated by reference herein in its entirety.

More particularly, high stringency hybridization conditions, as referred to herein, refer to conditions which permit isolation of nucleic acid molecules having at least about 75% nucleic acid sequence identity with the nucleic acid molecule being used to probe in the hybridization reaction, more particularly at least about 85%, and most particularly at least about 95%. Such conditions will vary, depending on whether DNA:RNA or DNA:DNA hybrids are being formed. Calculated melting temperatures for DNA:DNA hybrids are 10° C. less than for DNA:RNA hybrids. In particular embodiments, stringent hybridization conditions for DNA:DNA hybrids include hybridization at an ionic strength of 0.1×SSC (0.157 M Na⁺) at a temperature of between about 20° C. and about 35° C., more preferably, between about 28° C. and about 40° C., and even more preferably, between about 35° C. and about 45° C. In particular embodiments, stringent hybridization conditions for DNA:RNA hybrids include hybridization at an ionic strength of 0.1×SSC (0.157 M Na⁺) at a temperature of between about 30° C. and about 45° C., more preferably, between about 38° C. and about 50° C., and even more preferably, between about 45° C. and about 55° C. These values are based on calculations of a melting temperature for molecules larger than about 100 nucleotides, 0% formamide and a G+C content of about 50%. Alternatively, Tₘ can be calculated empirically as set forth in Sambrook et al., supra, pages 11.55 to 11.57.

Yet another agent that is useful in the methods of the present invention is a ribozyme. According to the present invention, a ribozyme typically contains stretches of complementary RNA bases that can base-pair with a target RNA ligand, including the RNA molecule itself, giving rise to an active site of defined structure that can cleave the bound RNA molecule (See Maulik et al., 1997, supra). Therefore, a ribozyme can serve as a targeting delivery vehicle for a nucleic acid molecule, or alternatively, the ribozyme can target and bind to RNA encoding IL-2 or IL-15, for example, and thereby effectively inhibit the translation of IL-2 or IL-15, respectively.

In some aspects of the present invention, the methods for increasing or decreasing memory T cell or CD25⁺ T cell responses further include administering to the patient an antigen against which the targeted immune response is generated. One embodiment of the present invention relates to a vaccine which includes a vaccinating antigen, or immunogen, and the combination of an agent that increases the activity of IL-15 and an agent that decreases the activity of IL-2 (the combination of which is referred to as a vaccine adjuvant).

According to the present invention, the terms “immunogen”, “vaccinating antigen”, and “antigen” can be used interchangeably, although the term “antigen” is primarily used herein to describe a protein which elicits a humoral and/or cellular immune response (i.e., is antigenic) under any suitable conditions, and the terms “immunogen” and “vaccinating antigen” are primarily used herein to describe a protein which elicits a humoral and/or cellular immune response in vivo, such that administration of the immunogen to an animal mounts an immunogen-specific (antigen-specific) immune response against the same or similar proteins that are encountered within the tissues of the animal. According to the present invention, an immunogen or an antigen can be any portion of a protein, naturally occurring or synthetically derived, which elicits a humoral and/or cellular immune response. As such, the size of an antigen or immunogen can be as small as about 5-12 amino acids and as large as a full length protein, including a multimer and fusion proteins. The terms, “immunogen” and “antigen”, as used to describe the present invention, can include a superantigen. A superantigen is defined herein as the art-recognized term. More particularly, a superantigen is a molecule within a family of proteins that binds to the extracellular portion of an MHC molecule (i.e., not in the peptide binding groove) to form and MHC:superantigen complex. The activity of a T cell can be modified when a TCR binds to an MHC:superantigen complex. Under certain circumstances, an MHC:superantigen complex can have a mitogenic role (i.e., the ability to stimulate the proliferation of T cells) or a suppressive role (i.e., deletion of T cell subsets). In preferred embodiments, the immunogen includes at least a portion of a tumor antigen or an antigen of an infectious disease pathogen (i.e., a pathogen antigen). As used herein, “at least a portion of an immunogen” (or antigen) refers to a portion of an immunogen containing a T cell and/or a B cell epitope. It is noted that an antigen can be provided in the form of a recombinant nucleic acid molecule encoding the antigen, if this type of delivery is desired.

In one aspect, the immunogen includes at least portion of a tumor antigen from a cancer selected from the group of melanomas, squamous cell carcinoma, breast cancers, head and neck carcinomas, thyroid carcinomas, soft tissue sarcomas, bone sarcomas, testicular cancers, prostatic cancers, ovarian cancers, bladder cancers, skin cancers, brain cancers, angiosarcomas, hemangiosarcomas, mast cell tumors, primary hepatic cancers, lung cancers, pancreatic cancers, gastrointestinal cancers, renal cell carcinomas, hematopoietic neoplasias and metastatic cancers thereof.

In another aspect, the immunogen includes at least a portion of an antigen from an infectious disease pathogen that can include pathogen antigens having epitopes that are recognized by T cells, pathogen antigens having epitopes that are recognized by B cells, pathogen antigens that are exclusively expressed by pathogens, and pathogen antigens that are expressed by pathogens and by other cells. Preferably, pathogen antigens useful in the present method have at least one T cell and/or B cell epitope and are exclusively expressed by pathogens (i.e., and not by the endogenous tissues of the infected mammal). According to the present invention, a pathogen antigen includes an antigen that is expressed by a bacterium, a virus, a parasite, a fungus, or any other pathogenic microorganism or organism. Preferred pathogen antigens for use in the method of the present invention include antigens which cause a chronic infectious disease in a mammal. For example, pathogen antigens for use in the present method can include immunogens from immunodeficiency virus (HIV), Mycobacterium tuberculosis, herpesvirus, papillomavirus and Candida.

The present invention includes the use of various compositions comprising combinations of the agents for increasing or decreasing the activity of IL-2 or IL-15 as described above. A vaccine is a specific type of composition that is used to elicit an immune response against a particular antigen (i.e., an immunogen), or group of antigens (e.g., several different antigens contained within a pathogenic organism or several different antigens chosen to be administered together). Adjuvants are agents that are capable of enhancing the immune response of an animal to a specific antigen. One embodiment of the invention relates to a vaccine adjuvant that includes: (a) an agent that increases interleukin-15 (IL-15) activity; and, (b) an agent that decreases interleukin-2 (IL-2) activity. Agents suitable for use in the vaccine adjuvant have been described in detail above. Such a vaccine adjuvant can include a vaccinating antigen, or immunogen, to form a vaccine, as well as one or more pharmaceutically acceptable carriers (described below), if desired.

In general, a composition of the present invention includes at least one agent that increases or decreases the activity of IL-15 and at least one agent having the opposite effect (i.e., decreases or increases, respectively) on the activity of IL-2, although in some aspects of the invention, agents acting on only one of the cytokines might be used. A composition can, in some embodiments, include an immunogen as discussed above, and typically, a composition includes a pharmaceutically acceptable carrier, which includes pharmaceutically acceptable excipients and/or delivery vehicles, for delivering the agent(s) to a patient. According to the present invention, a “pharmaceutically acceptable carrier” includes pharmaceutically acceptable excipients and/or pharmaceutically acceptable delivery vehicles, which are suitable for use in administration of the composition to a suitable in vitro, ex vivo or in vivo site. A suitable in vitro, in vivo or ex vivo site is the site of delivery of the composition of the present invention, including a vaccination site, the site of a tumor, the site of an autoimmune reaction, and/or a specific tissue or cell (e.g., a tumor cell, a graft cell, a memory T cell, a CD25⁺ T cell). Preferred pharmaceutically acceptable carriers are capable of maintaining a protein, antibody, small molecule, or recombinant nucleic acid molecule useful in the present invention in a form that, upon arrival of the protein, antibody, small molecule, or recombinant nucleic acid molecule at the cell target in a culture or in patient, the protein, antibody, small molecule, or recombinant nucleic acid molecule is capable of interacting with its target (e.g., a cell).

Suitable excipients of the present invention include excipients or formularies that transport or help transport, but do not specifically target a composition to a cell (also referred to herein as non-targeting carriers). Examples of pharmaceutically acceptable excipients include, but are not limited to water, phosphate buffered saline, Ringer's solution, dextrose solution, serum-containing solutions, Hank's solution, other aqueous physiologically balanced solutions, oils, esters and glycols. Aqueous carriers can contain suitable auxiliary substances required to approximate the physiological conditions of the recipient, for example, by enhancing chemical stability and isotonicity.

Suitable auxiliary substances include, for example, sodium acetate, sodium chloride, sodium lactate, potassium chloride, calcium chloride, and other substances used to produce phosphate buffer, Tris buffer, and bicarbonate buffer. Auxiliary substances can also include preservatives, such as thimerosal, m- or o-cresol, formalin and benzol alcohol. Compositions of the present invention can be sterilized by conventional methods and/or lyophilized.

One type of pharmaceutically acceptable carrier includes a controlled release formulation that is capable of slowly releasing a composition of the present invention into a patient or culture. As used herein, a controlled release formulation comprises an agent of the present invention (e.g., a protein (including homologues), an antibody, a nucleic acid molecule, or a mimetic) in a controlled release vehicle. Suitable controlled release vehicles include, but are not limited to, biocompatible polymers, other polymeric matrices, capsules, microcapsules, microparticles, bolus preparations, osmotic pumps, diffusion devices, liposomes, lipospheres, and transdermal delivery systems. Other carriers of the present invention include liquids that, upon administration to a patient, form a solid or a gel in situ. Preferred carriers are also biodegradable (i.e., bioerodible).

A pharmaceutically acceptable carrier which is capable of targeting can be referred to as a “delivery vehicle” or more particularly, a “targeting delivery vehicle.” Delivery vehicles of the present invention are capable of delivering a composition of the present invention to a target site in a patient. A “target site” refers to a site in a patient to which one desires to deliver a composition (e.g., a memory T cell, a CD25⁺ T cell, a tumor site/cell, a site of an autoimmune response, a vaccination site, a tissue/cell graft). For example, a target site can be any cell which is targeted by direct injection or delivery using liposomes, viral vectors or other delivery vehicles, including ribozymes. A cell or tissue can be targeted, for example, by including in the vehicle a targeting moiety, such as a ligand capable of selectively (i.e., specifically) binding another molecule at a particular site (i.e., a molecule on the surface of the target cell or a molecule expressed by cells in the target tissue/organ). Examples of such ligands include antibodies, antigens, receptors and receptor ligands. Alternatively, particular modes of administration (e.g., direct injection) and/or types of delivery vehicles (e.g., liposomes) can be used to deliver a composition preferentially to a particular site (see, for example, the use of cationic liposomes by intravenous delivery to target pulmonary tissues, described below). By way of example, a memory T cell can be targeted by its expression of CD44 or IL-2Rβ, for example, or more generally by targeting any suitable T cell surface molecule (e.g., CD3, TcR, CD4, CD8). Similarly, a site of an autoimmune response can be targeted by using a ligand that binds to a cell surface autoantigen or to a cell surface molecule that is expressed by a particular cell type or tissue type in proximity to the autoimmune response. Molecules to be targeted will be apparent to those of skill in the art.

Examples of delivery vehicles include, but are not limited to, artificial and natural lipid-containing delivery vehicles, viral vectors, and ribozymes. Natural lipid-containing delivery vehicles include cells and cellular membranes. Artificial lipid-containing delivery vehicles include liposomes and micelles. A delivery vehicle of the present invention can be modified to target to a particular site in a mammal, thereby targeting and making use of a compound of the present invention at that site. Suitable modifications include manipulating the chemical formula of the lipid portion of the delivery vehicle and/or introducing into the vehicle a compound capable of specifically targeting a delivery vehicle to a preferred site, for example, a preferred cell type. Specifically, targeting refers to causing a delivery vehicle to bind to a particular cell by the interaction of the compound in the vehicle to a molecule on the surface of the cell. Suitable targeting compounds include ligands capable of selectively (i.e., specifically) binding another molecule at a particular site. Examples of such ligands include antibodies, antigens, receptors and receptor ligands. Manipulating the chemical formula of the lipid portion of the delivery vehicle can modulate the extracellular or intracellular targeting of the delivery vehicle. For example, a chemical can be added to the lipid formula of a liposome that alters the charge of the lipid bilayer of the liposome so that the liposome fuses with particular cells having particular charge characteristics. Other suitable delivery vehicles include gold particles, poly-L-lysine/DNA-molecular conjugates, and artificial chromosomes.

In one embodiment, an agent of the present invention is targeted to a target site by using an antibody that selectively binds to a protein expressed on the surface of the target cell. For example, an antibody could bind to a tumor cell antigen or to an autoantigen. Such an antibody can include functional antibody equivalents such as antibody fragments (antigen binding fragments) (e.g., Fab fragments or Fab₂ fragments) and genetically-engineered antibodies, including single chain antibodies or chimeric antibodies, including bi-specific antibodies that can bind to more than one epitope. Such targeting antibodies are complexed with an agent that increases or decreases the activity of IL-2 or IL-15 action of the cell or in the local environment of the cell that is targeted, and serves to deliver the agent to the preferred site of action. The antibodies can be complexed to the target by any suitable means, including by complexing with a liposome, or by recombinant or chemical linkage of the agent to the antibody. In one embodiment, the agent is a second antibody or portion thereof that forms a chimeric or bispecific antibody with the targeting antibody.

When the agent is a nucleic acid molecule, a host cell is preferably transfected in vivo (i.e., in a mammal) as a result of administration to an animal of a recombinant nucleic acid molecule, or ex vivo, by removing cells from the animal and transfecting the cells with a recombinant nucleic acid molecule ex vivo. Transfection of a nucleic acid molecule into a host cell according to the present invention can be accomplished by any method by which a nucleic acid molecule administered into the cell in vivo or ex vivo, and includes, but is not limited to, transfection, electroporation, microinjection, lipofection, adsorption, viral infection, naked DNA injection and protoplast fusion. Methods of administration are discussed in detail below.

It may be appreciated by one skilled in the art that use of recombinant DNA technologies can improve expression of transfected nucleic acid molecules by manipulating, for example, the duration of expression of the gene (i.e., recombinant nucleic acid molecule), the number of copies of the nucleic acid molecules within a host cell, the efficiency with which those nucleic acid molecules are transcribed, the efficiency with which the resultant transcripts are translated, and the efficiency of post-translational modifications. Recombinant techniques useful for increasing the expression of nucleic acid molecules of the present invention include, but are not limited to, operatively linking nucleic acid molecules to high-copy number plasmids, integration of the nucleic acid molecules into one or more host cell chromosomes, addition of vector stability sequences to plasmids, increasing the duration of expression of the recombinant molecule, substitutions or modifications of transcription control signals (e.g., promoters, operators, enhancers), substitutions or modifications of translational control signals (e.g., ribosome binding sites, Shine-Dalgamo sequences), modification of nucleic acid molecules of the present invention to correspond to the codon usage of the host cell, and deletion of sequences that destabilize transcripts. The activity of an expressed recombinant protein of the present invention may be improved by fragmenting, modifying, or derivatizing nucleic acid molecules encoding such a protein.

In one embodiment of the present invention, a recombinant nucleic acid molecule useful in the present invention is administered to a patient in a liposome delivery vehicle, whereby the nucleic acid sequence encoding the protein enters the host cell (i.e., the target cell) by lipofection. A liposome delivery vehicle contains the recombinant nucleic acid molecule and delivers the molecules to a suitable site in a host recipient. According to the present invention, a liposome delivery vehicle comprises a lipid composition that is capable of delivering a recombinant nucleic acid molecule of the present invention, including both plasmids and viral vectors, to a suitable cell and/or tissue in a patient. A liposome delivery vehicle of the present invention comprises a lipid composition that is capable of fusing with the plasma membrane of the target cell to deliver the recombinant nucleic acid molecule into a cell.

A liposome delivery vehicle of the present invention can be modified to target a particular site in a mammal (i.e., a targeting liposome), thereby targeting and making use of a nucleic acid molecule of the present invention at that site. Suitable modifications include manipulating the chemical formula of the lipid portion of the delivery vehicle. Manipulating the chemical formula of the lipid portion of the delivery vehicle can elicit the extracellular or intracellular targeting of the delivery vehicle. For example, a chemical can be added to the lipid formula of a liposome that alters the charge of the lipid bilayer of the liposome so that the liposome fuses with particular cells having particular charge characteristics. Other targeting mechanisms include targeting a site by addition of exogenous targeting molecules (i.e., targeting agents) to a liposome (e.g., antibodies, soluble receptors or ligands). Suitable liposomes for use with the present invention include any liposome. Preferred liposomes of the present invention include those liposomes commonly used in, for example, gene delivery methods known to those of skill in the art. Complexing a liposome with a nucleic acid molecule of the present invention can be achieved using methods standard in the art.

In accordance with the present invention, acceptable protocols to administer an agent including the route of administration and the effective amount of an agent to be administered to an animal can be determined and executed by those skilled in the art. Effective dose parameters can be determined by experimentation using in vitro cell cultures, in vivo animal models, and eventually, clinical trials if the patient is human. Effective dose parameters can be determined using methods standard in the art for a particular disease or condition that the patient has or is at risk of developing. Such methods include, for example, determination of survival rates, side effects (i.e., toxicity) and progression or regression of disease.

Administration routes include in vivo, in vitro and ex vivo routes. In vivo routes include, but are not limited to, oral, nasal, intratracheal injection, inhaled, transdermal, rectal, and parenteral routes. Preferred parenteral routes can include, but are not limited to, subcutaneous, intradermal, intravenous, intramuscular and intraperitoneal routes. Preferred methods of in vivo administration include, but are not limited to, intravenous administration, intraperitoneal administration, intramuscular administration, intracoronary administration, intraarterial administration (e.g., into a carotid artery), subcutaneous administration, transdermal delivery, intratracheal administration, subcutaneous administration, intraarticular administration, intraventricular administration, inhalation (e.g., aerosol), intracerebral, nasal, oral, pulmonary administration, impregnation of a catheter, and direct injection into a tissue. Intravenous, intraperitoneal, intradermal, subcutaneous and intramuscular administrations can be performed using methods standard in the art. Aerosol (inhalation) delivery can also be performed using methods standard in the art (see, for example, Stribling et al., Proc. Natl. Acad. Sci. USA 189:11277-11281, 1992, which is incorporated herein by reference in its entirety). Oral delivery can be performed by complexing a therapeutic composition of the present invention with a carrier capable of withstanding degradation by digestive enzymes in the gut of an animal. Examples of such carriers, include plastic capsules or tablets, such as those known in the art. Direct injection techniques are particularly useful for suppressing graft rejection by, for example, injecting the composition into the transplanted tissue, or for site-specific administration of an agent, such as at the site of a tumor. Administration of a composition locally within the area of a target cell/tissue (e.g., transplanted tissue or tumor) refers to injecting or otherwise introducing the composition centimeters and preferably, millimeters within the target cell/tissue. Such routes can include the use of pharmaceutically acceptable carriers as described above.

Ex vivo refers to performing part of the regulatory step outside of the patient, such as by transfecting a population of cells removed from a patient with a recombinant molecule comprising a nucleic acid sequence encoding IL-2 or IL-15 according to the present invention under conditions such that the recombinant molecule is subsequently expressed by the transfected cell, or contacting a cell with another agent useful in the invention, and returning the transfected/contacted cells to the patient. In vitro and ex vivo routes of administration of a composition to a culture of host cells can be accomplished by a method including, but not limited to, transfection, transformation, electroporation, microinjection, lipofection, adsorption, protoplast fusion, use of protein carrying agents, use of ion carrying agents, use of detergents for cell permeabilization, and simply mixing (e.g., combining) a compound in culture with a target cell.

Various methods of administration and delivery vehicles disclosed herein have been shown to be effective for delivery of a nucleic acid molecule to a target cell, whereby the nucleic acid molecule transfected the cell and was expressed. In many studies, successful delivery and expression of a heterologous gene was achieved in preferred cell types and/or using preferred delivery vehicles and routes of administration of the present invention. All of the publications discussed below and elsewhere herein with regard to gene delivery and delivery vehicles are incorporated herein by reference in their entirety. For example, using liposome delivery, U.S. Pat. No. 5,705,151, issued Jan. 6, 1998, to Dow et al. demonstrated the successful in vivo intravenous delivery of a nucleic acid molecule encoding a superantigen and a nucleic acid molecule encoding a cytokine in a cationic liposome delivery vehicle, whereby the encoded proteins were expressed in tissues of the animal, and particularly in pulmonary tissues. As discussed above, Liu et al., 1997, ibid. demonstrated that intravenous delivery of cholesterol-containing cationic liposomes containing genes preferentially targets pulmonary tissues and effectively mediates transfer and expression of the genes in vivo. Several publications by Dzau and collaborators demonstrate the successful in vivo delivery and expression of a gene into cells of the heart, including cardiac myocytes and fibroblasts and vascular smooth muscle cells using both naked DNA and Hemagglutinating virus of Japan-liposome delivery, administered by both incubation within the pericardium and infusion into a coronary artery (intracoronary delivery) (See, for example, Aoki et al., 1997, J. Mol. Cell, Cardiol. 29:949-959; Kaneda et al., 1997, Ann N.Y. Acad. Sci. 811:299-308; and von der Leyen et al., 1995, Proc Natl Acad Sci USA 92:1137-1141). Delivery of numerous nucleic acid sequences has been accomplished by administration of viral vectors encoding the nucleic acid sequences. Using such vectors, successful delivery and expression has been achieved using ex vivo delivery (See, of many examples, retroviral vector; Blaese et al., 1995, Science 270:475-480; Bordignon et al., 1995, Science 270:470-475), nasal administration (CFTR-adenovirus-associated vector), intracoronary administration (adenoviral vector and Hemagglutinating virus of Japan, see above), intravenous administration (adeno-associated viral vector; Koeberl et al., 1997, Proc Natl Acad Sci USA 94:1426-1431). A publication by Maurice et al., 1999, ibid. demonstrated that an adenoviral vector encoding a β2-adrenergic receptor, administered by intracoronary delivery, resulted in diffuse multichamber myocardial expression of the gene in vivo, and subsequent significant increases in hemodynamic function and other improved physiological parameters. Levine et al. describe in vitro, ex vivo and in vivo delivery and expression of a gene to human adipocytes and rabbit adipocytes using an adenoviral vector and direct injection of the constructs into adipose tissue (Levine et al., 1998, J. Nutr. Sci. Vitaminol. 44:569-572). Gene delivery to synovial lining cells and articular joints has had similar successes. Oligino and colleagues report the use of a herpes simplex viral vector which is deficient for the immediate early genes, ICP4, 22 and 27, to deliver and express two different receptors in synovial lining cells in vivo (Oligino et al., 1999, Gene Ther. 6:1713-1720). The herpes vectors were administered by intraarticular injection. Kuboki et al. used adenoviral vector-mediated gene transfer and intraarticular injection to successfully and specifically express a gene in the temporomandibular joints of guinea pigs in vivo (Kuboki et al., 1999, Arch. Oral. Biol. 44:701-709). Apparailly and colleagues systemically administered adenoviral vectors encoding IL-10 to mice and demonstrated successful expression of the gene product and profound therapeutic effects in the treatment of experimentally induced arthritis (Apparailly et al., 1998, J. Immunol. 160:5213-5220). In another study, murine leukemia virus-based retroviral vector was used to deliver (by intraarticular injection) and express a human growth hormone gene both ex vivo and in vivo (Ghivizzani et al., 1997, Gene Ther. 4:977-982). This study showed that expression by in vivo gene transfer was at least equivalent to that of the ex vivo gene transfer. As discussed above, Sawchuk et al. has reported successful in vivo adenoviral vector delivery of a gene by intraarticular injection, and prolonged expression of the gene in the synovium by pretreatment of the joint with anti-T cell receptor monoclonal antibody (Sawchuk et al., 1996, ibid. Finally, it is noted that ex vivo gene transfer of human interleukin-1 receptor antagonist using a retrovirus has produced high level intraarticular expression and therapeutic efficacy in treatment of arthritis, and is now entering FDA approved human gene therapy trials (Evans and Robbins, 1996, Curr. Opin. Rheumatol. 8:230-234). Therefore, the state of the art in gene therapy has led the FDA to consider human gene therapy an appropriate strategy for the treatment of at least arthritis. Taken together, all of the above studies in gene therapy indicate that delivery and expression of a cytokine-encoding recombinant nucleic acid molecule according to the present invention is feasible.

Another method of delivery of recombinant molecules is in a non-targeting carrier (e.g., as “naked” DNA molecules, such as is taught, for example in Wolff et al., 1990, Science 247, 1465-1468). Such recombinant nucleic acid molecules are typically injected by direct or intramuscular administration. Recombinant nucleic acid molecules to be administered by naked DNA administration include a nucleic acid molecule of the present invention, and preferably includes a recombinant molecule of the present invention that preferably is replication, or otherwise amplification, competent.

According to the method of the present invention, an effective amount of an agent that regulates IL-2 or IL-15 to administer to an animal comprises an amount that is capable of regulating IL-2 or IL-15 activity, and preferably effecting a modulation of an immune response at a target site, without being toxic to the animal. An amount that is toxic to an animal comprises any amount that causes damage to the structure or function of an animal (i.e., poisonous). A preferred single dose of an agent typically comprises between about 0.01 microgram×kilogram⁻¹ and about 10 milligram×kilogram⁻¹ body weight of an animal. A more preferred single dose of an agent comprises between about 1 microgram×kilogram⁻¹ and about 10 milligram×kilogram⁻¹ body weight of an animal. An even more preferred single dose of an agent comprises between about 5 microgram×kilogram⁻¹ and about 7 milligram×kilogram⁻¹ body weight of an animal. An even more preferred single dose of an agent comprises between about 10 microgram×kilogram⁻¹ and about 5 milligram×kilogram⁻¹ body weight of an animal. A particularly preferred single dose of an agent comprises between about 0.1 milligram×kilogram⁻¹ and about 5 milligram×kilogram⁻¹ body weight of an animal, if the an agent is delivered by aerosol. Another particularly preferred single dose of an agent comprises between about 0.1 microgram×kilogram⁻¹ and about 10 microgram×kilogram⁻¹ body weight of an animal, if the agent is delivered parenterally. These doses particularly apply to the administration of protein agents, antibodies, and/or small molecules (i.e., the products of drug design). Preferably, a protein or antibody of the present invention is administered in an amount that is between about 50 U/kg and about 15,000 U/kg body weight of the patient. When the compound to be delivered is a nucleic acid molecule, an appropriate single dose results in at least about 1 pg of protein expressed per mg of total tissue protein per μg of nucleic acid delivered. More preferably, an appropriate single dose is a dose which results in at least about 10 pg of protein expressed per mg of total tissue protein per μg of nucleic acid delivered; and even more preferably, at least about 50 pg of protein expressed per mg of total tissue protein per μg of nucleic acid delivered; and most preferably, at least about 100 pg of protein expressed per mg of total tissue protein per μg of nucleic acid delivered. A preferred single dose of a naked nucleic acid vaccine ranges from about 1 nanogram (ng) to about 100 μg, depending on the route of administration and/or method of delivery, as can be determined by those skilled in the art.

The methods of the present invention can be used in any animal, and particularly, in any animal of the Vertebrate class, Mammalia, including, without limitation, primates, rodents, livestock and domestic pets. Preferred mammals to treat using the method of the present invention include humans.

Yet another embodiment of the present invention relates to a method to identify a compound that increases memory T cell responses. Such a compound is preferably an agonist of IL-15, but not of IL-2. In one aspect, the compound is an antagonist of IL-2. In another aspect, such a compound is identified as being an agonist of IL-15 and an antagonist of IL-2. The method includes the steps of contacting a putative regulatory compound (i.e., putative agonist or antagonist) with an IL-15 receptor and an IL-2 receptor, and identifying compounds that have IL-15 agonist activity, IL-2 antagonist activity, or both activities. The step of identifying more specifically includes determining whether the compound increases memory T cell responses. In a preferred embodiment, the method includes a step of identifying an agonist of IL-15 and an antagonist of IL-2 (i.e., identifying two compounds for use together in a therapeutic method of the present invention).

Another embodiment of the invention relates to a method to identify a compound that inhibits undesirable T cell responses (e.g., autoimmune responses). Such a compound is preferably an agonist of IL-2, but not of IL-15. In one aspect, the compound is an antagonist of IL-15. In another aspect, such a compound is identified as being an agonist of IL-2 and an antagonist of IL-15. The method includes the steps of contacting a putative regulatory compound (i.e., putative agonist or antagonist) with an IL-15 receptor and an IL-2 receptor, and identifying compounds that have IL-2 agonist activity, IL-15 antagonist activity, or both activities. The step of identifying more specifically includes determining whether the compound inhibits undesirable T cell responses and/or binds to and activates CD25⁺ regulatory T cells. In a preferred embodiment, the method includes a step of identifying an agonist of IL-2 and an antagonist of IL-15 (i.e., identifying two compounds for use together in a therapeutic method of the present invention).

As used herein, the phrase “IL-15 agonist” or “IL-2 agonist” refers to any compound that interacts with an IL-15 receptor or an IL-2 receptor, respectively, and elicits an observable response. More particularly, an agonist can include, but is not limited to, a protein, peptide, antibody, or any suitable product of drug design/screening (i.e., non-peptide drug), that selectively binds to and activates or increases the activation of the IL-15 or IL-2 receptor, respectively, and that is characterized by its ability to agonize (e.g., stimulate, induce, increase, enhance) the biological activity of a naturally occurring IL-15 or IL-2 receptor in a manner similar to the natural agonist, IL-15 or IL-2, respectively (e.g., by interaction/binding with and/or direct or indirect activation of the receptor). It is noted that the effect of the action of a given agonist on the expression of a downstream event may be the downregulation of the event or the suppression of the event. However, the term agonist is intended to refer to the ability of the putative ligand to act on a receptor in a manner that is substantially similar to the action of the natural receptor ligand (e.g., IL-15 or IL-2) on the receptor. Typically, an agonist is identified under conditions wherein, in the absence of the agonist, the receptor is not activated, or is at least believed not to be in the presence of a compound that is known to activate the receptor, such as the natural ligand.

The phrase, “IL-15 antagonist” or “IL-2 antagonist” refers to any compound which inhibits the effect of an IL-15 or IL-2 agonist, respectively, as described above. More particularly, an antagonist is capable of associating with a receptor such that the biological activity of the receptor is decreased (e.g., reduced, inhibited, blocked, reversed, altered) in a manner that is antagonistic (e.g., against, a reversal of, contrary to) to the action of the natural agonist on the receptor. Such a compound can include, but is not limited to, a protein, peptide, antibody, or product of drug design/screening that selectively binds to and blocks access to the receptor by a natural or synthetic agonist ligand or reduces or inhibits the activity of a receptor. It is noted that the action of a given antagonist on a given downstream event via the receptor may be to actually upregulate the event. However, the term antagonist is intended to refer to the ability of the ligand to act on a receptor in a manner that is antagonistic to the action of the natural ligand on the receptor. Typically, an antagonist is identified under control conditions wherein, in the absence of the antagonist, the receptor is stimulated, such as by the natural ligand, or by any suitable known agonist. In one embodiment, an antagonist can be identified by its ability to alter the regulation of downstream events by the receptor in the absence of a known stimulator of the receptor.

Agonists and antagonists that are products of drug design can be produced using various methods known in the art. Various methods of drug design, useful to design mimetics or other regulatory compounds useful in the present invention are disclosed in Maulik et al., 1997, Molecular Biotechnology: Therapeutic Applications and Strategies, Wiley-Liss, Inc., which is incorporated herein by reference in its entirety. Such methods have been discussed previously herein.

As used herein, the term “putative” refers to compounds having an unknown or previously unappreciated regulatory activity in a particular process. As such, the term “identify” is intended to include all compounds, the usefulness of which as a regulatory compound of IL-2 or IL-15 receptor activation for the purposes of increasing memory T cell responses or decreasing undesirable T cell responses is determined by a method of the present invention.

In the method of identifying a compound that increases memory T cell responses or decreases undesirable T cell responses, the method can be a cell-based assay, or non-cell-based assay. In one embodiment, the IL-2 and/or IL-15 receptor is expressed by a cell (i.e., a cell-based assay). In another embodiment the IL-2 and/or IL-15 receptor is in a cell lysate, or is purified or produced free of cells (e.g., a soluble IL-2 and/or IL-15 receptor). In accordance with the present invention, a cell-based assay is conducted under conditions which are effective to screen for regulatory compounds useful in the method of the present invention. Effective conditions include, but are not limited to, appropriate media, temperature, pH and oxygen conditions that permit cell growth. An appropriate, or effective, medium refers to any medium in which a cell of the present invention, when cultured, is capable of cell growth and expression of an IL-2 and/or IL-15 receptor. Such a medium is typically a solid or liquid medium comprising growth factors and assimilable carbon, nitrogen and phosphate sources, as well as appropriate salts, minerals, metals and other nutrients, such as vitamins. Culturing is carried out at a temperature, pH and oxygen content appropriate for the cell. Such culturing conditions are within the expertise of one of ordinary skill in the art.

In one embodiment, the conditions under which a receptor according to the present invention is contacted with a putative regulatory compound, such as by mixing, are conditions in which the receptor is not stimulated (activated) if essentially no regulatory compound is present. For example, such conditions include normal culture conditions in the absence of a stimulatory compound (a stimulatory compound being, e.g., the natural ligand for the receptor, a stimulatory antibody, or other equivalent stimulus). In this embodiment, the putative regulatory compound is then contacted with the receptor. The step of detecting or identifying is designed to indicate whether the putative regulatory compound binds to the IL-2 and/or IL-15 receptor, and further, whether the putative regulatory compound stimulates the receptor, and further, whether the putative regulatory compound increases memory T cell responses, inhibits undesirable T cell responses, and/or increases the activity of CD25⁺ regulatory T cells.

The present methods involve contacting cells with the compound being tested for a sufficient time to allow for interaction, activation or inhibition of the receptor by the compound. The cells can naturally express the IL-2 and/or IL-15 receptor, or can recombinantly express an IL-2 and/or IL-15 receptor functional unit. The period of contact with the compound being tested can be varied depending on the result being measured, and can be determined by one of skill in the art. For example, for binding assays, a shorter time of contact with the compound being tested is typically suitable, than when activation is assessed. As used herein, the term “contact period” refers to the time period during which cells are in contact with the compound being tested. The term “incubation period” refers to the entire time during which cells are allowed to grow prior to evaluation, and can be inclusive of the contact period. Thus, the incubation period includes all of the contact period and may include a further time period during which the compound being tested is not present but during which growth is continuing (in the case of a cell based assay) prior to scoring. The incubation time for growth of cells can vary but is sufficient to allow for the binding of the receptor, activation of the receptor, and/or inhibition of the receptor. It will be recognized that shorter incubation times are preferable because compounds can be more rapidly screened. A preferred incubation time is between about 1 minute to about 48 hours.

The assay of the present invention can also be a non-cell based assay. In this embodiment, the putative regulatory compound can be directly contacted with an isolated receptor, or a receptor component (e.g., an isolated extracellular portion of the receptor, or soluble receptor), and the ability of the putative regulatory compound to bind to the receptor or receptor component can be evaluated, such as by an immunoassay or other binding assay (competitive binding techniques, equilibrium dialysis or BIAcore methods). The assay can then include the step of further analyzing whether putative regulatory compounds which bind to a portion of the receptor are capable of increasing or decreasing the activity of the IL-2 and/or IL-15 receptor. Such further steps can be performed by cell-based assay, as described above.

The method of identifying a regulatory agent (compound) additionally includes detecting or identifying whether the putative regulatory agent activates the receptor in a manner that increases or decreases memory T cell growth and proliferation and/or increases or decreases the activity of CD25⁺ T cells. IL-15 and IL-2 agonists can be identified in a straightforward matter by their ability to support the growth of an IL-2-dependent cell line (e.g., HT-2) or to increase the proliferation of a T cell line that expresses the IL-2R and/or the IL-15R. In particular, the IL-15 agonists can be identified by their ability to bind to an IL-15R and to support the growth and proliferation of memory T cells in any in vitro assay. Similarly, IL-15 antagonists can be identified by their ability to bind to an IL-15 receptor, and by their inability to support the growth and proliferation of memory T cells in an in vitro assay, particularly as compared to IL-15. IL-2 agonists can be identified by their ability to bind to and support the growth and proliferation of CD25⁺ T cells and/or to kill memory T cells. IL-2 antagonists can be identified by their ability to bind to an IL-2 receptor, and by their inability to support the growth and proliferation of CD25⁺ T cells and/or to kill memory T cells.

The following examples are provided for the purpose of illustration and are not intended to limit the scope of the present invention.

### EXAMPLES

### Example 1

The following example demonstrates that memory CD8⁺ T cells are CD44^{high} and IL-₂Rβ^{high}.

To investigate the causes of memory T cell division, memory T cells were characterized and a system was established in which the phenomenon could be studied. Memory T cells bear high levels of CD44 (Picker et al., J. Immunol., 145:3247 (1990); Swain and Bradley, Semin. Immunol. 4:59 (1992); Griffin and Orme, Infect. Immunol. 62:1683 (1994)) and high levels of IL72 receptor β (IL-2Rβ), a polypeptide that is shared by the receptors for IL-2 and IL-15 (Cho et al., Proc. Natl. Acad. Sci. USA 96:2976 (1999); Nelson and Willerford, Adv. Immunol. 70:1 (1998)). To confirm this phenotype, the levels of IL-2Rβ on CD44^{low} and CD44^{high} CD8+ T cells from normal young or old mice, or on antigen primed T cells bearing a transgenic T cell receptor (TCR) specific for K^{b} bound to a peptide from ovalbumin (Hogquist et al., Cell 76:17 (1994)) were measured.

More specifically, PBL were isolated from C57B1/6 mice, stained with anti-CD8, anti-IL-2Rβ and anti CD44 and analyzed. T cells were isolated and stained and analyzed as described in P. Marrack, J. Kappler, T. Mitchell, J. Exp. Med. 189, 521 (1999), incorporated by reference in its entirety, using antibodies from PharMingen, San Diego, Calif. Cells were labeled with CFSE (Molecular Probes, Oreg.) by the method of S. A. Weston, C. R. Parish J. Immunol. Methods 133, 87 (1990), incorporated by reference in its entirety. Incorporation of BrdU into cellular DNA was measured as described by P. Carayon, A. Bord J. Immunol. Methods 147, 225 (1992), incorporated by reference in its entirety, using anti-BrdU (Becton Dickinson, San Jose, Calif.). Mice were purchased from The Jackson Laboratory, Bar Harbor, Me., or from the National Institute of Aging mouse colony at Charles River Laboratories, Willington, Mass.

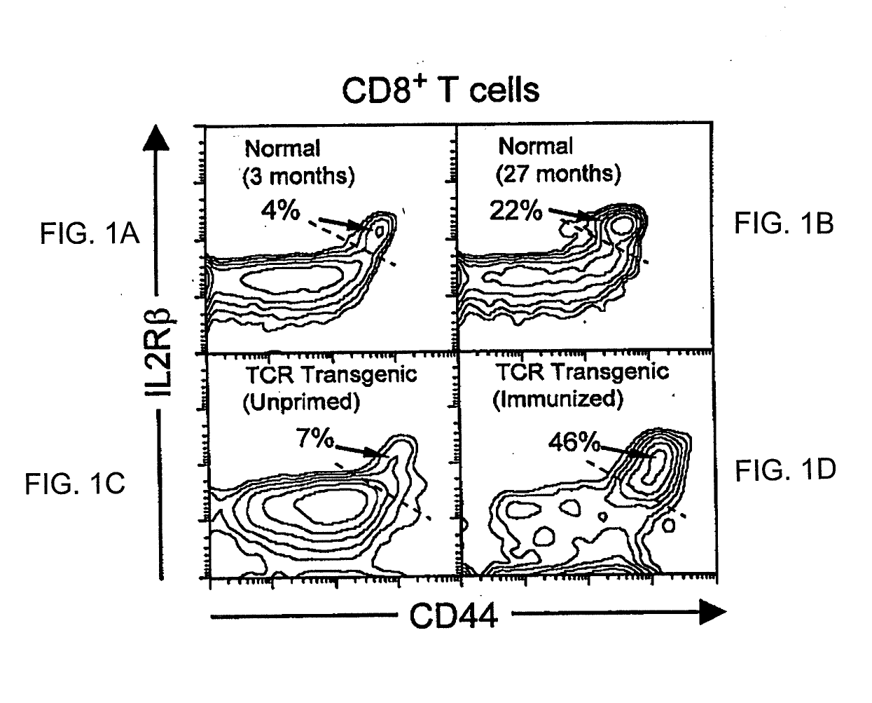

C57B1/6 mice transgenenic for the OTI TCR (Hogquist et al., Cell 76:17 (1994)) were untreated (FIG. 1C) or infected with Vaccinia Virus modified to express chicken ovalbumin (FIG. 1D). Forty seven days later T cells were purified by passage over nylon wool, stained and analyzed as described above, except that cells were also gated to be Vα2+.

The results demonstrated that almost all of the CD8+ cells that bear high levels of CD44 also bear high levels of IL-2Rβ and vice versa (FIG. 1). Also, as expected, the proportion of CD8+ T cells that were IL-2Rβ^{high}, CD44^{high} increased as the animals aged (Barrat et al., Res. Immunol. 146:23 (1995); Miller et al., FASEB J. 10:775 (1997)). In young mice, exposure to antigen converted CD44^{low}, IL-2Rβ^{low} TCR transgenic, CD8+ T cells into CD44^{high}, IL-2Rβ^{high} cells. These experiments confirmed that both environmentally created and deliberately primed memory CD8+ T cells were CD44^{high} and IL-2Rβ^{high}.

### Example 2

The following example demonstrates that CD8+ T cells of memory phenotype divide slowly.

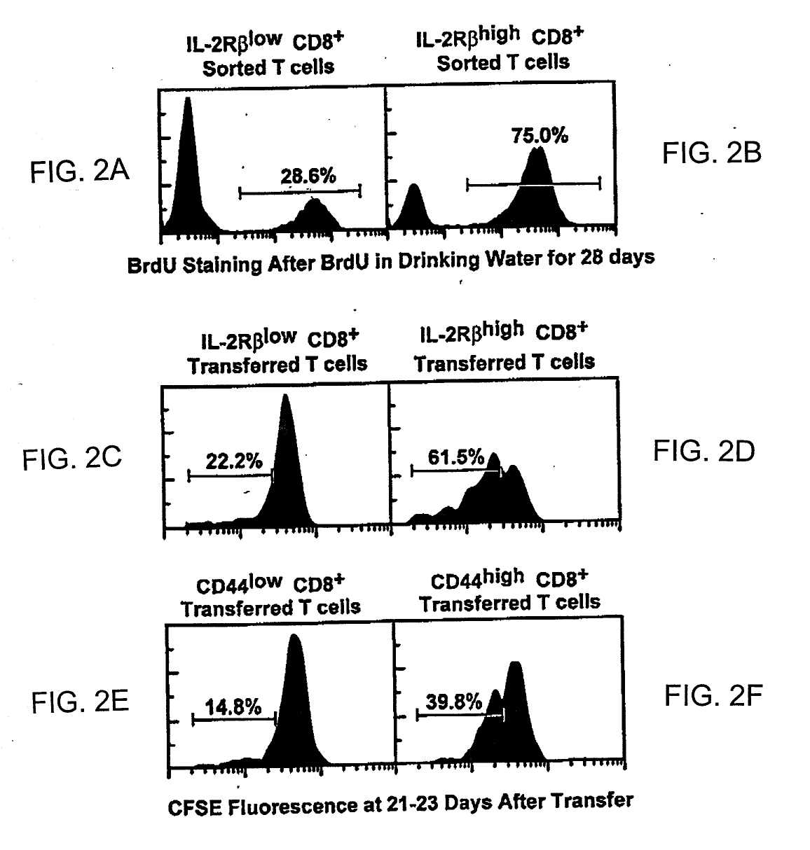

Memory T cells are thought to divide slowly in animals (Bruno et al., Eur. J. Immunol. 26:3179 (1996); Murali-Krishna et al., Science 286:1377 (1999); Swain et al., Science 286:1381 (1999)). To confirm this, mice were given BrdU in their drinking water for 28 days. CD8+ T cells from the mice were then analyzed for incorporation of BrdU into their DNA, an indication of cell division. Briefly, C57B1/10 mice were thymectomized when they were 8 weeks old. Five weeks later 0.8 mg/ml BrdU was added to their drinking water for 28 days. Their T cells were then purified, stained with anti-IL-2Rβ and anti-CD8 and sorted into CD8+ populations bearing low or high amounts of IL-2Rβ. The sorted cells were stained with anti-BrdU (See Example 1 above). Anti-BrdU staining of the cells bearing low (FIG. 2A) and high (FIG. 2B) amounts of IL-2RP is shown in FIGS. 2A and 2B. In addition, T cells were isolated from 10 month old C57BL/6 mice, stained with anti-IL-2Rβ and anti-CD8 and sorted into CD8+ cells bearing low or high amounts of IL-2Rβ or CD44. The sorted populations were stained with CFSE and transferred into nonirradiated, 12 week old syngeneic recipients. 21-23 days later T cells were purified from the recipients and analyzed for CFSE staining. Data shown in FIGS. 2C-2F are for transferred CD8+ cells which were IL-2Rβ^{low} (FIG. 2C), IL-2Rβ^{high} (FIG. 2D), CD44^{low} (FIG. 2E) or CD44^{high} (FIG. 2F).

The data in FIGS. 2A and 2B show that more of the IL-2Rβ^{high} CD8+ T cells had divided than the IL-2Rβ^{low} cells. To find out how frequently the cells were dividing, IL-2Rβ^{high} or IL-2Rβ^{low} or CD44hi′ or CD44^{low} CD8+ T cells were sorted, labeled with CFSE and transferred into normal recipients. Many more of the IL-2Rβ^{high} or CD₄₄^{high} cells divided than did their IL-2Rβ^{low} or CD44^{low} counterparts, as demonstrated by dilution of their CFSE stain (FIGS. 2C-2F). These experiments confirm that, in animals, CD8+ T cells of memory phenotype divide slowly. Previous experiments by others and the present inventors' data suggest that this division is antigen independent since it occurs in β2microglobulin deficient (β2MKO) mice (3,10, see Example 3).

### Example 3

The following example shows that IL-15 drives the proliferation of memory CD8+ T cells and that IL-2 causes the death of the dividing cells.

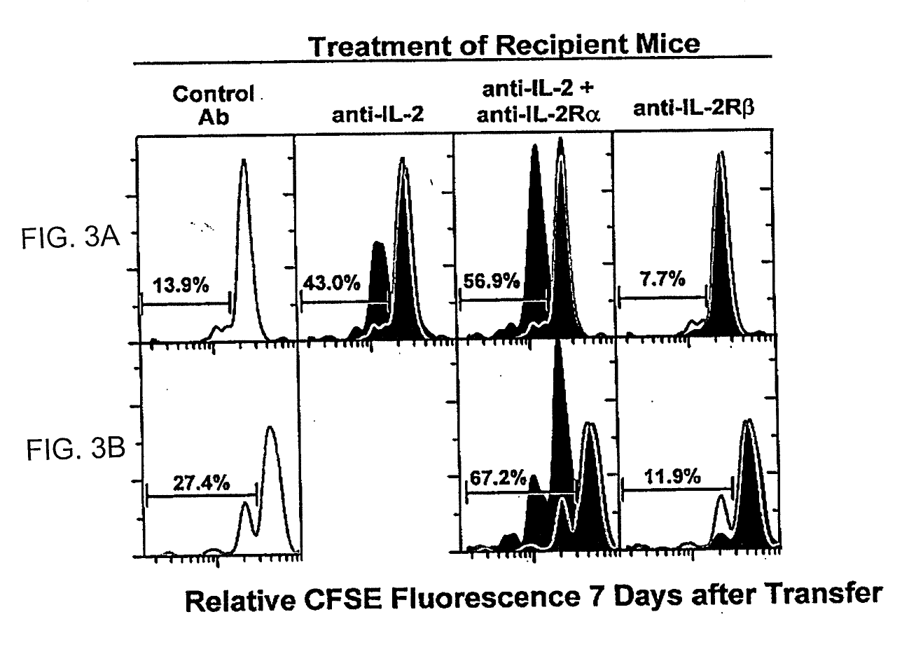

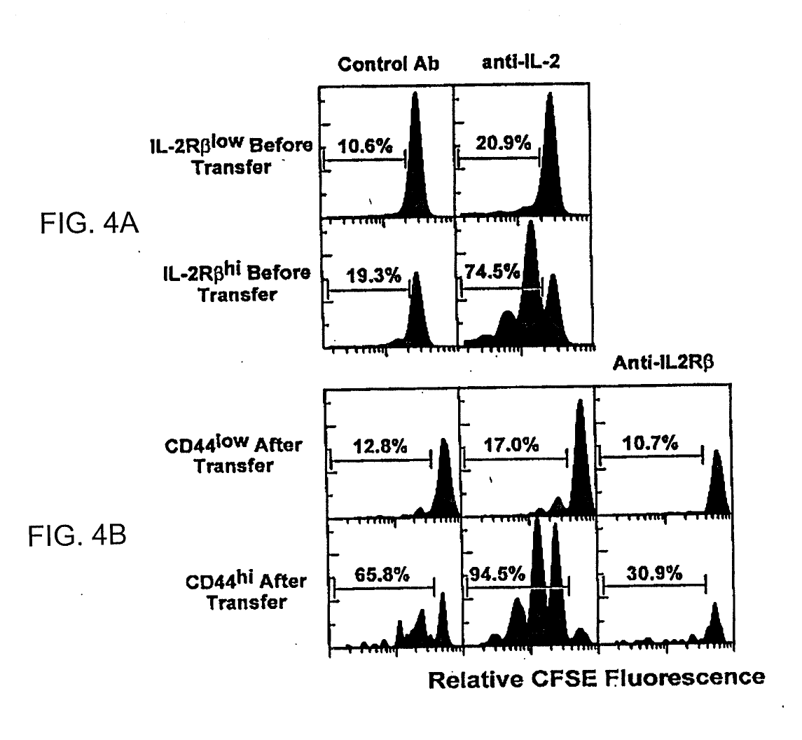

To investigate the idea that cytokines might be driving this proliferation, a number of antibodies against cytokines and cytokine receptors were tested for their ability to affect the process. Anti-IL-2Rβ was used to block signaling by IL-2 and/or IL-15, since the receptors for these cytokines share the IL-2Rβ chain (Nelson and Willerford, Adv. Immunol. 70:1 (1998)). To distinguish between the effects of IL-2 and IL-15, the results with anti-IL-2Rβ were compared with those with anti-IL-2 (sometimes combined with anti-IL-2Rα), which blocks IL-2 but not IL-15. These are all rat antibodies so normal rat IgG was used as a control. To prevent Fc mediated effects of the anti-receptor antibodies, they were converted to F(ab′)2's. Since the recipients would eventually respond to the rat antibodies, the duration of the experiment was limited to 7-9 days. Examples of these experiments are shown in FIGS. 3 and 4 and the results are summarized in Table 1.

In FIG. 3, unseparated CFSE labeled T cells were transferred and analyzed. Briefly, T cells were isolated from the lymph nodes and spleens of: a 12 month old adult thymectomized mouse (FIG. 3A) and a 26 month old C57BL/6 mouse (FIG. 3B), labeled with CFSE and transferred into nonirradiated, 8 week old, syngeneic animals. On days 2-6 after transfer the animals were injected intraperitoneally with 1 mg/day of the indicated antibodies and F(ab′)₂'s. Seven days after transfer, T cells were isolated from the recipients, stained with anti-CD8 and anti-TCR Cβ or antibody to the Vβ expressed on large CD8+ clones known to be in the mice and analyzed. Data shown are for the Cβ+, CD8+, CFSE+ cells, or, if the mice were >12 months old, for CD8+, CFSE+ cells excluding large CD8+ clones. The light line represents the CFSE staining of cells from mice treated with control Ab. The shaded areas represent the CFSE profiles of T cells from animals treated with the indicated antibodies. Percentages on the Figure represent the percentages of the surviving CFSE labeled cells which had divided.

In the short time of the experiment, proliferation was modest in control mice but effects of the antibodies could still be seen. Anti-IL-2Rβ consistently inhibited proliferation of the transferred cells. This was due to effects on IL-15, rather than on IL-2 because anti-IL-2, with or without anti-IL-2Rα dramatically increased the numbers of dividing cells. This was not due to crosslinking of the IL-2R by anti-IL-2Rα since anti-IL-2 was effective alone. Anti-IL-2 was sometimes not as effective as the combination of anti-IL-2 plus anti-IL-2Rα, probably because of less efficient blocking of IL-2. The results of a number of experiments of this type are summarized in Table 1. Anti-IL-2 with or without anti-IL-2Rα always substantially increased the number of proliferating CD8+ T cells while anti-IL-2Rβ decreased the number. None of these treatments had any effect on expression of IL-2Rα by the transferred cells.

TABLE 1
The Rate of Appearance of Dividing CD8 + T Cells
Is Increased By IL-15 And Reduced By IL-2.

| | % of Control Proliferation of | | |
|---|---|---|---|
| | Transferred Cells in Mice Treated with: | | |
| Donor Age | | Anti-IL-2 + | |
| (months) | Anti-IL-2 | Anti-IL-2Rα | Anti-IL-2Rβ |
| 3 | 458 | | |
| 3 | 469 | | |
| 6 | 337 | | |
| 10 | 189 | | |
| 10 | 187 | | |
| 12 | 354 | 505.2 | 51.7 |
| | | | 47.4 |
| 12 | | 476.9 | 36.8 |
| 18 | 177.5 | | 25.5 |

The experiments were performed as described in FIG. 3 and the text. Cells analyzed were alive, CFSE+ and CD8+. The %s of recovered, transferred T cells which had divided during the course of the experiments in mice receiving control Ab ranged from 11.6% to 37.3%. The % of control proliferation of transferred cells in experimental mice was calculated as the % of the surviving, transferred T cells which divided in mice treated with the indicated antibodies divided by the % of the surviving, transferred T cells which divided in mice treated with control Ab.

Since most of the proliferating cells were of memory phenotype (FIG. 2), it was likely that these were the cells affected by the antibody treatments. To demonstrate this directly, purified populations of naive and memory phenotype cells were transferred (FIG. 4A) or naive versus memory phenotype cells were gated at the time of analysis (FIG. 4B). Briefly, T cells from 12 week old C57BL/10 mice were purified, stained with anti-CD8 and anti-IL-2Rβ, sorted into CD8+, IL-2Rβ^{low} and CD8+, IL-2Rβ^{high} populations and transferred into 12 week old, nonirradiated syngeneic recipients (FIG. 4A). The recipients were treated with antibody as described for FIG. 3. Seven days after transfer T cells were isolated stained and analyzed as described for FIG. 3. In the experiment shown in FIG. 4B, T cells from 18 month old thymectomized C57B1/6 mice were treated as described in FIG. 3 except that they were transferred into 12 week old recipients. Mice were given antibodies for 7 days and sacrificed 9 days after cell transfer. Cells were stained with anti-CD44 and CD44^{low} and CD44^{high} cells analyzed separately.

In both cases, most of the proliferating cells were of memory phenotype. Recovery of these proliferating memory phenotype cells was greatly stimulated by anti-IL-2 and greatly inhibited by anti-IL-2Rβ. Similar effects have been seen in preliminary experiments with memory T cells produced by deliberate priming with antigen.

To confirm the previous report that this proliferation by CD8+ memory T cells was not driven by antigen, in a preliminary experiment, β2MKO T cells were developed in β2M sufficient chimeras, primed with vaccinia virus, CFSE labeled, and transferred to β2MKO nonirradiated hosts. The hosts were then treated with control rat Ig or anti-IL-2. Seven days after transfer, 9.4% of the transferred CD8+, IL-2Rβ^{high} cells had divided in the rat Ig treated host, whereas an average of 59.6% of the transferred CD8+, IL-2Rβ^{high} cells had divided in anti-IL-2 treated recipients.

Whether the cells were transferred into normal or β2MKO recipients, little proliferation by naive cells was observed (FIG. 4 and data not shown). This small amount of proliferation was marginally stimulated by anti-IL-2 and blocked by anti-IL-2Rβ, results consistent with a small contamination of the naive cells by memory cells.

To find out if these treatments affected the total numbers of cells, the numbers of transferred CD8+ T cells of memory phenotype recovered per mouse from animals treated with the various antibodies were calculated. In this experiment, anti-IL-2Rβ dramatically reduced the numbers of cells which had divided and had no effect on the numbers of cells which had not divided. Conversely, anti-IL-2 treatment increased the yield of dividing cells tremendously, and had a modest effect on the yield of nondividing cells. These results show that IL-15 drives the proliferation of memory CD8+ T cells and that IL-2 causes the death of the dividing cells, rather than inhibition of division of the precursors. Altogether, these data show that IL-15 and IL-2 have a profound effect on the total numbers of CD8+ memory phenotype cells in the animals. Even in the short term of this experiment, lack of IL-15 caused a drop by one third in the total numbers of transferred CD8+ memory phenotype cells, and lack of IL-2 caused an increase of more than ten fold in the size of this same population.

TABLE 2
IL-15 increases and IL-2 decreases the total numbers of memory
phenotype CD8+ T cells in animals by affecting dividing cells.

| Numbers of Donor Memory Phenotype CD8+ | | |
|---|---|---|
| T Cells/Recipient × 10⁻³ (% Control) | | |
| Dividing | Nondividing | Total |

| | | | | | | |
|---|---|---|---|---|---|---|
| Control | 8.0 | | 8.1 | | 16.1 | |
| Anti-IL-2 | 170 | (2125) | 22 | (275) | 192 | (1193) |

Transfer and analyses were done as described in FIG. 4B. Cell numbers are for CD44high, CD8+ T cells in the spleens and inguinal, axillary, brachial, superficial cervical, mesenteric, lumbar and caudal lymph nodes of recipients.

While various embodiments of the present invention have been described in detail, it is apparent that modifications and adaptations of those embodiments will occur to those skilled in the art. It is to be expressly understood, however, that such modifications and adaptations are within the scope of the present invention, as set forth in the following claims.

What is claimed:

1. A vaccine adjuvant, comprising:
a. an agent that increases interleukin-15 (IL-15) activity; and,
b. an agent that decreases interleukin-2 (IL-2) activity.

2. The vaccine adjuvant of claim 1, wherein said agent that increases IL-15 activity is an agent that increases IL-15 receptor activity without enhancing IL-2 receptor activity.

3. The vaccine adjuvant of claim 2, wherein said agent that increases IL-15 activity is IL-15 or a homologue of IL-15 that has IL-15 biological activity.

4. The vaccine adjuvant of claim 2, wherein said agent that increases IL-15 activity is an antibody that selectively binds to and activates an IL-15 receptor and does not substantially bind to and activate an IL-2 receptor.

5. The vaccine adjuvant of claim 2, wherein said agent selectively binds to IL-15Rα.

6. The vaccine adjuvant of claim 1, wherein said agent that increases IL-15 activity is an agent that binds to and increases the half-life of IL-15.

7. The vaccine adjuvant of claim 1, wherein said agent that increases IL-15 activity is a recombinant nucleic acid molecule comprising a nucleic acid sequence encoding IL-15 or a homologue of IL-15 that has IL-15 biological activity.

8. The vaccine adjuvant of claim 1, wherein said agent that increases IL-15 activity is an agent that binds to a regulatory region of a gene encoding IL-15 and increases transcription of said gene encoding IL-15.

9. The vaccine adjuvant of claim 1, wherein said agent that decreases IL-2 activity is an antibody that selectively binds to IL-2 and blocks IL-2, eliminates IL-2 or prevents the interaction of IL-2 with its receptor.

10. The vaccine adjuvant of claim 1, wherein said agent that decreases IL-2 activity is a compound that binds to and degrades IL-2.

11. The vaccine adjuvant of claim 1, wherein said agent that decreases IL-2 activity is a compound that blocks or decreases the activity of IL-2 receptors without blocking or decreasing the activity of IL-15 receptors.

12. The vaccine adjuvant of claim 11, wherein said agent selectively binds to IL-2Rα.

13. The vaccine adjuvant of claim 1, wherein said agent that decreases IL-2 activity is an antisense nucleic acid molecule that hybridizes to a gene encoding IL-2 under high stringency conditions and inhibits the expression of IL-2.

14. The vaccine adjuvant of claim 1, further comprising a delivery vehicle that targets memory T lymphocytes.

15. The vaccine adjuvant of claim 14, wherein said delivery vehicle comprises an antibody that selectively binds to a cell surface molecule expressed by memory T lymphocytes.

16. A vaccine comprising:
a. the vaccine adjuvant of claim 1; and
b. a vaccinating antigen.

17. The vaccine of claim 16, wherein said vaccinating antigen is selected from the group consisting of: a tumor antigen and an antigen from an infectious disease pathogen.

18. A method to increase T lymphocyte memory against an antigen, comprising administering to an animal the vaccine of claim 16.

19. A method to increase T lymphocyte memory comprising administering to an animal a composition comprising an agent that increases IL-15 activity and an agent that decreases IL-2 activity.

20. The method of claim 19, wherein said step of administering increases the activity or survival of CD25⁺ T cells in said animal.

21. The method of claim 19, wherein said composition is administered to a site of a vaccination in said animal.

22. The method of claim 19, further comprising administering to said animal an antigen against which T lymphocyte memory is to be induced.

23. The method of claim 19, wherein said agent that increases IL-15 activity is IL-15 or a homologue of IL-15 that has IL-15 biological activity.

24. The method of claim 19, wherein said agent that increases IL-15 activity is an antibody that selectively binds to and activates an IL-15 receptor and does not substantially bind to and activate an IL-2 receptor.

25. The method of claim 24, wherein said agent selectively binds to IL-15Rα.

26. The method of claim 19, wherein said agent that increases IL-15 activity is an agent that binds to and increases the half-life of IL-15.

27. The method of claim 19, wherein said agent that increases IL-15 activity is a recombinant nucleic acid molecule comprising a nucleic acid sequence encoding IL-15 or a homologue of IL-15 that has IL-15 biological activity.

28. The method of claim 19, wherein said agent that increases IL-15 activity is an agent that binds to a regulatory region of a gene encoding IL-15 and increases transcription of said gene encoding IL-15.

29. The method of claim 19, wherein said agent that decreases IL-2 activity is an antibody that selectively binds to IL-2 and blocks IL-2, eliminates IL-2 or prevents the interaction of IL-2 with its receptor.

30. The method of claim 19, wherein said agent that decreases IL-2 activity is a compound that binds to and degrades IL-2.

31. The method of claim 19, wherein said agent that decreases IL-2 activity is a compound that blocks or decreases the activity of IL-2 receptors without blocking or decreasing the activity of IL-15 receptors.

32. The method of claim 31, wherein said agent selectively binds to IL-2Rα.

33. The method of claim 19, wherein said agent that decreases IL-2 activity is an antisense nucleic acid molecule that hybridizes to a gene encoding IL-2 under high stringency conditions and inhibits the expression of IL-2.

34. A method to reduce an autoimmune response, comprising administering to a site of autoimmune response a composition comprising an agent that increases the activity of IL-2.

35. The method of claim 34, wherein said agent that increases IL-2 activity is an agent that increases IL-2 receptor activity without enhancing IL-15 receptor activity.

36. The method of claim 35, wherein said agent that increases IL-2 activity is IL-2 or a homologue of IL-2 that has IL-2 biological activity.

37. The method of claim 35, wherein said agent that increases IL-2 activity is an antibody that selectively binds to and activates an IL-2 receptor and does not substantially bind to and activate an IL-15 receptor.

38. The method of claim 35, wherein said agent selectively binds to IL-2Rα.

39. The method of claim 34, wherein said agent that increases IL-2 activity is an agent that binds to and increases the half-life of IL-2.

40. The method of claim 34, wherein said agent that increases IL-2 activity is a recombinant nucleic acid molecule comprising a nucleic acid sequence encoding IL-2 or a homologue of IL-2 that has IL-2 biological activity.

41. The method of claim 34, wherein said agent that increases IL-2 activity is an agent that binds to a regulatory region of a gene encoding IL-2 and increases transcription of said gene encoding IL-2.

42. The method of claim 34, further comprising administering to said site of said autoimmune response an agent that decreases IL-15 activity.

43. The method of claim 42, wherein said agent that decreases IL-15 activity is an antibody that selectively binds to IL-15 and blocks IL-15, eliminates IL-15 or prevents the interaction of IL-15 with its receptor.

44. The method of claim 42, wherein said agent that decreases IL-15 activity is a compound that binds to and degrades IL-15.

45. The method of claim 42, wherein said agent that decreases IL-15 activity is a compound that blocks or decreases the activity of IL-15 receptors without blocking or decreasing the activity of IL-2 receptors.

46. The method of claim 45, wherein said agent selectively binds to IL-15Rα.

47. The method of claim 42, wherein said agent that decreases IL-15 activity is an antisense nucleic acid molecule that hybridizes to a gene encoding IL-15 under high stringency conditions and inhibits the expression of IL-15.

48. The method of claim 34, wherein said composition comprises a delivery vehicle that selectively targets a site of an autoimmune response.

49. The method of claim 48, wherein said delivery vehicle comprises an antibody that selectively binds to a cell surface molecule expressed by a cell at said site of said autoimmune response.

50. The method of claim 34, wherein said composition further comprises an autoantigen against which said autoimmune response is directed.

51. A composition for decreasing an undesirable T cell response, comprising:
a. an agent that increases the activity of IL-2; and
b. an agent that decreases the activity of IL-15.
